SEQUENCE LISTING (65 sequences)

SEQ ID NO: 1 - protein, 32 aa, Artificial
  TALENs nuclease
NNNNHDHDNNNNHDNIHDHDNIHDNINNHDHD

SEQ ID NO: 2 - protein, 36 aa, Artificial
  TALENs nuclease
NGNINININNHDNINGNNNGNGNNNNNGHDNGNNNG

SEQ ID NO: 3 - protein, 32 aa, Artificial
  TALENs nuclease
NNHDNGNGNGNININININNHDHDNGHDNINI

SEQ ID NO: 4 - protein, 36 aa, Artificial
  TALENs nuclease
NNHDHDNGNGNGHDHDNGNGHDHDHDNGNNHDHDNI

SEQ ID NO: 5 - protein, 36 aa, Artificial
  TALENs nuclease
NNNNHDHDNNNNHDNIHDHDNIHDNINNHDHDNIHD

SEQ ID NO: 6 - protein, 36 aa, Artificial
  TALENs nuclease
NGNGNINININNHDNINGNNNGNGNNNNNGHDNGNN

SEQ ID NO: 7 - protein, 36 aa, Artificial
  TALENs nuclease
NNHDNGNGNGNININININNHDHDNGHDNINININI

SEQ ID NO: 8 - protein, 36 aa, Artificial
  TALENs nuclease
NNNNNGNGNNHDHDNGNGNGHDHDNGNGHDHDHDNG

SEQ ID NO: 9 - protein, 36 aa, Artificial; identical to SEQ ID NO: 7
  TALENs nuclease

SEQ ID NO: 10 - protein, 36 aa, Artificial
  TALENs nuclease
NGNNNNNGNGNNHDHDNGNGNGHDHDNGNGHDHDHD

SEQ ID NO: 11 - protein, 34 aa, Artificial
  TALENs nuclease
NNNNHDHDNNNNHDNIHDHDNIHDNINNHDHDNI

SEQ ID NO: 12 - protein, 36 aa, Artificial
  TALENs nuclease
NINININNHDNINGNNNGNGNNNNNGHDNGNNNGNG

SEQ ID NO: 13 - protein, 36 aa, Artificial
  TALENs nuclease
HDNINNNIHDHDNINIHDNINGNNHDNGNGNGNINI

SEQ ID NO: 14 - protein, 32 aa, Artificial
  TALENs nuclease
HDHDNINGNGNNNININIHDNGNNNNHDNINN

SEQ ID NO: 15 - protein, 34 aa, Artificial
  TALENs nuclease
NNNGNNNNHDHDNIHDNNNIHDHDHDHDNININN

SEQ ID NO: 16 - protein, 34 aa, Artificial
  TALENs nuclease
HDHDNININNNNNNNGNGNGNINNHDNINNNINN

SEQ ID NO: 17 - protein, 34 aa, Artificial
  TALENs nuclease
HDNINNNININNNNHDNGNNHDNINNNGNGHDHD

SEQ ID NO: 18 - protein, 32 aa, Artificial
  TALENs nuclease
HDNNNGNNNNHDHDNIHDNIHDHDNNNGNNNG

SEQ ID NO: 19 - protein, 36 aa, Artificial
  TALENs nuclease
NNNNHDHDNIHDNNNIHDHDHDHDNININNNIHDNI

SEQ ID NO: 20 - protein, 34 aa, Artificial
  TALENs nuclease
NINGNGHDHDNININNNNNNNGNGNGNINNHDNI

SEQ ID NO: 21 - protein, 34 aa, Artificial
  TALENs nuclease
HDNININININIHDHDNINGNGNNNININIHDNG

SEQ ID NO: 22 - protein, 32 aa, Artificial
  TALENs nuclease
NNNIHDNGHDHDHDNGHDNGNNHDNGNGNNNN

SEQ ID NO: 23 - protein, 32 aa, Artificial
  TALENs nuclease
NGHDNNHDNGNNNNNIHDHDNININIHDNINN

SEQ ID NO: 24 - protein, 36 aa, Artificial
  TALENs nuclease
HDNININGNNNNNGNGNGNGNGNNNINNNNHDNGNG

SEQ ID NO: 25 - protein, 36 aa, Artificial
  TALENs nuclease
NININININNHDHDNGHDNININININIHDHDNING

SEQ ID NO: 26 - protein, 36 aa, Artificial
  TALENs nuclease
HDNGNNHDNGNGNNNNNGNGNNHDHDNGNGNGHDHD

SEQ ID NO: 27 - protein, 36 aa, Artificial
  TALENs nuclease
HDNININININIHDHDNINGNGNNNININIHDNGNN

SEQ ID NO: 28 - protein, 36 aa, Artificial
  TALENs nuclease
NNNIHDNGHDHDHDNGHDNGNNHDNGNGNNNNNGNG

SEQ ID NO: 29 - DNA, 49 bp, Bos taurus
ggccggcaccacagccacttcgctggaccaaacagaccaacatgcttta

SEQ ID NO: 30 - DNA, 47 bp, Bos taurus
gctttaaaagcctcaaaaaccattgaaactggcagggaaggaaaggc

SEQ ID NO: 31 - DNA, 50 bp, Bos taurus
ggccggcaccacagccacttcgctggaccaaacagaccaacatgctttaa

SEQ ID NO: 32 - DNA, 52 bp, Bos taurus
gctttaaaagcctcaaaaaccattgaaactggcagggaaggaaaggcaacca

SEQ ID NO: 33 - DNA, 52 bp, Bos taurus; identical to SEQ ID NO: 32

SEQ ID NO: 34 - DNA, 48 bp, Bos taurus
ggccggcaccacagccacttcgctggaccaaacagaccaacatgcttt

SEQ ID NO: 35 - DNA, 46 bp, Bos taurus
cagaccaacatgctttaaaagcctcaaaaaccattgaaactggcag

SEQ ID NO: 36 - DNA, 50 bp, Bos taurus
gtggccacgaccccaagacaaaacccccttgatctctgctaaacccttgg

SEQ ID NO: 37 - DNA, 46 bp, Bos taurus
cagaaggctgcagttccaagcctgaccaagacacggtgtggccacg

SEQ ID NO: 38 - DNA, 51 bp, Bos taurus
ggccacgaccccaagacaaaacccccttgatctctgctaaacccttggaat

SEQ ID NO: 39 - DNA, 52 bp, Bos taurus
caaaaaccattgaaactggcagggaaggaaaggcaaccaagcagagggagtc

SEQ ID NO: 40 - DNA, 49 bp, Bos taurus
tcgctggaccaaacagaccaacatgctttaaaagcctcaaaaaccattg

SEQ ID NO: 41 - DNA, 52 bp, Bos taurus
aaaagcctcaaaaaccattgaaactggcagggaaggaaaggcaaccaagcag

SEQ ID NO: 42 - DNA, 52 bp, Bos taurus; identical to SEQ ID NO: 39

SEQ ID NO: 43 - DNA, 18 bp, Bos taurus
gaggcttttaaagcatgt

SEQ ID NO: 44 - DNA, 90 bp, Artificial
  introgression template
ggccctgggcatggccggcaccacagccacttctctagaccaaacagaccaacatgtttaaaagcctcaaaaaccattgaaactggcagg

SEQ ID NO: 45 - DNA, 91 bp, Artificial
  introgression template
ggccctgggcatggccggcaccacagccacttcgctggaccaaacagaccaacatgctttaaaagcctcaaaaaccattgaaactggcagg

SEQ ID NO: 46 - DNA, 90 bp, Artificial
  introgression template
agcctgaccaagacacggtgtggccatgaccccaagactctagacccttgatctctgctaaacccttggaatacgtggagatccacaagg

SEQ ID NO: 47 - DNA, 90 bp, Artificial
  introgression template
agcctgaccaagacacggtgtggccacgaccccaagacaaaacccccttgatctctgctaaacccttggaatacgtggagatccacaagg

SEQ ID NO: 48 - DNA, 90 bp, Artificial
  introgression template
gcaccacagccacttcgctggaccaaacagaccaacatgctttaaaagcctcaaaaaccattgaaactggcagggaaggaaaggcaacca

SEQ ID NO: 49 - DNA, 90 bp, Artificial; identical to SEQ ID NO: 48
  introgression template

SEQ ID NO: 50 - DNA, 20 bp, Artificial
  Primer for RFLP analysis
accttacatgtctccaggcc

SEQ ID NO: 51 - DNA, 21 bp, Artificial
  Primer for RFLP analysis
gggacacctttgagtactcct

SEQ ID NO: 52 - DNA, 20 bp, Artificial
  Primer for RFLP analysis
accttacatgtctccaggcc

SEQ ID NO: 53 - DNA, 21 bp, Artificial
  Primer for RFLP analysis
gggacacctttgagtactcct

SEQ ID NO: 54 - DNA, 20 bp, Artificial
  Primer for RFLP analysis
accttacatgtctccaggcc

SEQ ID NO: 55 - DNA, 21 bp, Artificial
  Primer for RFLP analysis
gggacacctttgagtactcct

SEQ ID NO: 56 - DNA, 2387 bp, Bos taurus
ggcaaatgctgaggatactttccaagtgaaccctgagtgaacctctaatatatttatttcctgtggaaagaggaaggagccaacatgaaggaaaatgcagcatctagagtggttttcattttgctactttttctcagtgtcagccttctgaatggacagtcacctcctgaaaaacccaagctcgttaaatgtcggtctcctggaaaggaaacattcacctgctggtgggagcctggggcagatggaggacttcctaccaattacacgctgacttaccacaaggaaggagaaacactcatccatgaatgtccagactacaaaaccgggggccccaactcctgctactttagcaagaagcacacctccatatggaagatgtacgtcatcacagtaaacgccatcaaccagatgggaatcagttcctcggatccactttatgtgcacgtgacttacatagttgaaccagagcctcctgcaaacctgactttggaattaaaacatccagaagatagaaaaccatatctatggataaaatggtctccacccaccatgactgatgtaaaatctggttggttcattatccagtacgaaattcgattaaaacctgagaaagcaactgattgggagactcattttactctgaagcaaactcagcttaagattttcaacttatatccaggacaaaaataccttgtgcagattcgctgcaagccagaccatggatactggagtgagtggagcccagagagctccatccagatacctaatgacttcccagtgaaggacacaagcatgtggatctttgtggccatcctttctgctgtcatctgtttgattatggtctgggcagtggctttgaagggctatagcatggtgacctgcatcctcccaccagttccagggccaaaaataaaaggatttgatgttcatctgctggagaagggcaagtccgaagaacttctgcgagctctggaaagccaagacttcccccccacttctgactgcgaggacttgctgatggagttcatagaggtagatgactgtgaggaccagcagctgatgccacgcccctccaaagaacacacggagcaaggcgtgaagcccatgcacctggatcttgacagtgactctggccggggcagctgcgacagcccttcgctcttgtctgaaaagtgtgatgaacctcaggcccatccctccaagttccatactcccgagggccctgagaagctggagaatccggaaacaaaccttacatgtctccaggcccctcagagcacaagcgtggaaggcaaaatcccctattttctggccaatggacccaaatcttccacatggcctttcccgcagccccccagcctatacagccccagatattcttaccacaacattgctgacgtgtgtgagctggccctgggcatggccggcaccacagccacttcgctggaccaaacagaccaacatgctttaaaagcctcaaaaaccattgaaactggcagggaaggaaaggcaaccaagcagagggagtcagaaggctgcagttccaagcctgaccaagacacggtgtggccacgaccccaagacaaaacccccttgatctctgctaaacccttggaatacgtggagatccacaaggtcagccaagatggagtgctggctctgttcccaaaacaaaacgagaagtttggcgcccctgaagccagcaaggagtactcaaaggtgtcccgggtgacagatagcaacatcctggtattggtgccggatccgcaagcgcaaaacctgactctgttagaagaaccagccaagaaggccccgccagccctgccatagaatccagccaaggccgacctggctatctcccccacaaccccaggcaactgcagactccagttgggctggggactgggtcccgcaggttttatgcactcttgcagtgagagttatggaaggatgggttcaattgtgattttccttcagggaacactacagagtacgtgaaatgcactctaccagagagggctcaagaacagggttagaatgacactacccaactcccagttcactcttaattctctattttcaaccagttgcctctttgtccaacagctgattccagaacaaatcgttccatcttgtgtgatttgtagatttacttttttgctattagttgtcagattatatgttcaaagatataaaagcacattgcctagtattcttaagagacagtgccaataggtatataatctggaaaaggccttcatggtttcgtatgtgacagaggggtataagtcagtcaaaattgtttaccatgggaagatggtagataggagagaaatgccatgaaaaccactttgaagaccagttgcttaacctttgcactcctcttt

SEQ ID NO: 57 - protein, 581 aa, Bos taurus
MKENAASRVVFILLLFLSVSLLNGQSPPEKPKLVKCRSPGKETFTCWWEPGADGGLPTNYTLTYHKEGETLIHECPDYKTGGPNSCYFSKKHTSIWKMYVITVNAINQMGISSSDPLYVHVTYIVEPEPPANLTLELKHPEDRKPYLWIKWSPPTMTDVKSGWFIIQYEIRLKPEKATDWETHFTLKQTQLKIFNLYPGQKYLVQIRCKPDHGYWSEWSPESSIQIPNDFPVKDTSMWIFVAILSAVICLIMVWAVALKGYSMVTCILPPVPGPKIKGFDVHLLEKGKSEELLRALESQDFPPTSDCEDLLMEFIEVDDCEDQQLMPRPSKEHTEQGVKPMHLDLDSDSGRGSCDSPSLLSEKCDEPQAHPSKFHTPEGPEKLENPETNLTCLQAPQSTSVEGKIPYFLANGPKSSTWPFPQPPSLYSPRYSYHNIADVCELALGMAGTTATSLDQTDQHALKASKTIETGREGKATKQRESEGCSSKPDQDTVWPRPQDKTPLISAKPLEYVEIHKVSQDGVLALFPKQNEKFGAPEASKEYSKVSRVTDSNILVLVPDPQAQNLTLLEEPAKKAPPALP

SEQ ID NO: 58 - DNA, 206 bp, Bos taurus
gggcatggccggcaccacagccacttcgctggaccaaacagaccaacatgcttaaaagcctcaaaaaccattgaaactggcagggaaggaaaggcaaccaagcagggagtcagaaggctgcagttccaagcctgaccaagacacggtgtggccacgaccccaagacaaaacccccttgatctctgctaaacccttggaatacgtgg

SEQ ID NO: 59 - DNA, 83 bp, Artificial Sequence
  Sythesized introgression of SLICK1 into native bovine cells
cctgggcatggccggcaccacagccacttcgctggaccaaacagaccaacatgtttaaaagcctcaaaaaccattgaaactgg

SEQ ID NO: 60 - DNA, 83 bp, Artificial Sequence
  Sythesized introgression of SLICK1 into native bovine cells
ggacccgtaccggccgtggtgtcggtgaagcgacctggtttgtctggttgtacaaattttcggagtttttggtaactttgacc

SEQ ID NO: 61 - DNA, 92 bp, Bos taurus
aagcctgaccaagacacggtgtggccacgaccccaagacaaaacccccttgatctctgctaaacccttggaatacgtggagatccacaaggt

SEQ ID NO: 62 - DNA, 57 bp, Bos taurus
ccacttcgctggaccaaacagaccaacatgctttaaaagcctcaaaaaccattgaaa

SEQ ID NO: 63 - DNA, 57 bp, Bos taurus
ggtgaagcgacctggtttgtctggttgtacgaaattttcggagtttttggtaacttt

SEQ ID NO: 64 - protein, 581 aa, Bos taurus; identical to SEQ ID NO: 57

SEQ ID NO: 65 - DNA, 2387 bp, Bos taurus; identical to SEQ ID NO: 56

(19) United States
(12) Patent Application Publication
(10) Pub. No.: US 2019/0223417 A1
(43) Pub. Date: Jul. 25, 2019

(54) GENETICALLY MODIFIED ANIMALS HAVING INCREASED HEAT TOLERANCE

(71) Applicant: Recombinetics, Inc., Saint Paul, MN (US)

(72) Inventors: Tad S. Sonstegard, Centreville, MD (US); Daniel F. Carlson, Woodbury, MN (US); Scott C. Fahrenkrug, Minneapolis, MN (US)

(21) Appl. No.: 16/282,232

(22) Filed: Feb. 21, 2019

Related U.S. Application Data
(62) Division of application No. 15/270,901, filed on Sep. 20, 2016, now abandoned.
(60) Provisional application No. 62/327,115, filed on Apr. 25, 2016.
(60) Provisional application No. 62/221,444, filed on Sep. 21, 2015.

(51) Int. Cl. A01K 67/027; C12N 15/90; C07K 14/715

(52) U.S. Cl. CPC A01K 67/0275; C12N 15/907; A01K 2227/101; A01K 2217/072; A01K 2267/02; C07K 14/715

(57) ABSTRACT

Disclosed herein are genomically modified livestock animals and methods to provide them that express the SLICK phenotype. The animals disclosed herein express a truncated allele for the prolactin receptor (PRLR) gene. When expressed, the livestock animals produce a PRLR that is missing up to the terminal 148 amino acid (aa) residues of the protein all ranges and values within the explicitly stated range are contemplated: e.g., from 148 to 69. Animals expressing SLICK have superior thermoregulatory ability compared to non-slick animals and experience a less drastic depression in milk yield during the summer.

### CROSS REFERENCE TO RELATED APPLICATIONS

This application is a divisional application of U.S. patent application Ser. No. 15/270,901, filed Sep. 20, 2016, which claims priority to U.S. Provisional Applications No. 62/221,444 filed Sep. 21, 2015, and 62/327,115 filed Apr. 25, 2016 each of which is hereby incorporated by reference in their entirety.

### SEQUENCE LISTING

The instant application contains a Sequence Listing which has been submitted electronically in ASCII format and is hereby incorporated by reference in its entirety. Said ASCII copy, is named 53545_719_201_SL.txt and is 66,240 bytes in size.

### FIELD OF THE INVENTION

The invention is directed to livestock animals genetically modified to have greater heat tolerance by expressing the SLICK phenotype.

### BACKGROUND OF THE INVENTION

Livestock animals are raised worldwide. Global agriculture and animal husbandry practices mean that a few breeds of livestock have been developed and raised in large numbers worldwide for their desirable qualities. Cattle, in particular are raised in large herds for both milk and beef production. However, most popular breeds of cattle were originally developed in Europe. These breeds include Angus, Holstein Friesian, Hereford, Shorthorn, Charolais, Jersey, Galloway, Brown Swiss, Chianina, and Belgian Blue to name a few.

Heat tolerance in livestock animals is essential for raising healthy animals and maintaining them at their production capacity. In cattle, for example, being able to maintain a normal body temperature means that the animals are disease resistant, produce more milk and grow bigger and reproduce more prolifically with healthier calves than cattle that are not tolerant of heat stress. This is particularly true for livestock raised in tropical and subtropical climates.

“SLICK” is a mutation found in new world cattle including Senepol, Carora, Criollo Limonero and Romosinuano. The term “SLICK” was coined to refer to the cattle's' short, glossy hair. This phenotype also includes hair density, hair type and sweat gland density and thermoregulation efficiency. Cattle having the SLICK phenotype exhibit greatly increased abilities to thermoregulate in tropical environments and consequently experience considerably less stress in hot environments.

The “SLICK” mutation has been mapped to chromosome 20 of the cattle genome and codes for the prolactin receptor (PRLR). The gene has nine exons that code for a polypeptide of 581 amino acids. Previous research in Senepol cattle has shown that the phenotype results from a single base deletion in exon 10 (there is no exon 1, recognized exons are 2-10) that introduces a premature stop codon (p.Leu462) and loss of the terminal 120 amino acids from the receptor. This phenotype is referred to herein as SLICK1. Senepol cattle are extremely heat tolerant and have been crossed with many other cattle breeds to provide the benefit of heat tolerance. It would be desirable to confer traits including heat tolerance to other breeds of animal without sexual mating resulting in the loss of traits for which particular animal breeds are desired.

### SUMMARY OF THE INVENTION

Disclosed herein are precision bred, gene edited livestock animals and methods to provide them that express the slick phenotype. The animals disclosed herein express a truncated allele for the prolactin receptor (PRLR) gene. When expressed, the livestock animals produce a PRLR that is missing up to the terminal 148 amino acids (aa) residues of the protein. In some embodiments the animal expresses a protein that is truncated by 147 or 146 aa. In some cases, the animal is missing the terminal 121 aa. In some embodiments, the Livestock animal expresses a PRLR that is missing the terminal 69 aa and exhibits the SLICK phenotype. Artisans will immediately appreciate that all ranges and values within the explicitly stated range are contemplated: e.g., from 148 to 69. That is, any PRLR expressing as its last amino acid tyrosine at position 433 of the protein having the GenBank Accession No. AAA51417 (SEQ ID NO 64), translated from mRNA identified as having the Accession No NM_001039726. Animals expressing SLICK have superior thermoregulatory ability compared to non-slick animals and experience a less drastic depression in milk yield during the summer.

In various exemplary embodiments, the disclosure provides a livestock animal genetically modified to express a modified prolactin receptor (PRLR) gene resulting in a truncated PRLR. In some embodiments, the PRLR is truncated after the tyrosine at residue 433 of the residue identified by GenBank Accession No. AAA51417. In various embodiments, the PRLR is truncated after the residue at AA 461, 496 or 464. In these exemplary embodiments, the livestock animal is less susceptible to heat stress. In various exemplary embodiments the animal is an artiodactyl. In some exemplary embodiments the artiodactyl is a bovine. In various exemplary embodiments the genetic modifications made by precision gene editing is made by nonmeiotic introgression gene editing using zinc finger nuclease, meganuclease, TALENs or CRISPR/CAS technology. In some exemplary embodiments, the genetic modification is heterozygous. In other exemplary embodiments, the genetic modification is homozygous. In some exemplary embodiments, the PRLR gene is modified following residue 1383 of the mRNA as identified by GenBank Accession No. NM_001039726 (SEQ ID NO 65). In various exemplary embodiments, the modification results in a break in the protein synthesis of the gene. In these exemplary embodiments, the animal expresses the SLICK phenotype.

In yet other exemplary embodiments, the disclosure provides a livestock animal genetically modified to express a SLICK phenotype comprising modification of the PRLR gene after residue 1383 as identified by the mRNA having GenBank accession No. NM_001039726. In various embodiments, the modification is made by nonmeiotic introgression gene editing using zinc finger nuclease, meganuclease, TALENs or CRISPR/CAS technology. In some exemplary embodiments, the genetic modification results in a PRLR having between 433 amino acids and 511 amino acids as identified by GenBank Accession No. AAA51417. In these exemplary embodiments, the genetic modification results in a PRLR protein having 433 amino acids, 461 amino acids, 464 amino acids, 496 amino acids, 511 amino acids or residues terminating between 433 amino acids and 511 amino acids. In various exemplary embodiments, the modification is made to a somatic cell and the animal is cloned by nuclear transfer from the somatic cell to an enucleated egg. In some exemplary embodiments, the modification comprises a mutation that breaks protein synthesis by providing in a deletion, insertion or mutation of the genetic reading frame.

In still yet other exemplary embodiments, the disclosure provides a method of genetically modifying livestock animals to express a SLICK phenotype comprising, expressing a prolactin receptor (PRLR) gene modified to break synthesis of the prolactin receptor (PRLR) protein after amino acid residue 433 as identified by GenBank Accession No. AAA51417 by using precision gene editing technologies including zinc finger nuclease, meganuclease, TALENs or CRISPR/CAS technology and a homology directed repair (HDR) template homologous to a portion of the PRLR designed to introduce a frame shift mutation or stop codon. In these exemplary embodiments, the break in synthesis is introduced after nucleotide 1383 of mRNA identified by GenBank accession No. NM_001039726. In some embodiments, the disclosure further includes introducing a nuclease restriction site proximate to the genetic modification. In various embodiments, the nuclease restriction site is downstream from the genetic modification. In other embodiments, the introduction of the nuclease restriction site are directed by the same HDR template. In various exemplary embodiments, the genetic modification and the introduction of the nuclease restriction site are directed by different HDR templates.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be apparent from the description, as set forth hereinafter.

### BRIEF DESCRIPTION OF THE DRAWINGS

Various exemplary embodiments of the compositions and methods according to the invention will be described in detail, with reference to the following figures wherein:

FIG. 1 is a cartoon of the prolactin receptor (PRLR) showing various isoforms of the peptide. The wt receptor is a dimer with each monomer having a total length of 581 aa. Naturally occurring isoforms of the peptide are shown. The transmembrane region is represented by the horizontal bi-lipid structure across the center of the figure. The extracellular domain is represented by the area above the transmembrane region and, the intracellular domain is the area below the transmembrane region. The slick phenotype is found in 3 breeds of cattle each having a different isoform of the PRLR. Slick I expressed by the Senepol breed have one monomer truncated at aa 461, e.g., a loss of the final 120 aa. SLICK2 expressed by Carora/Limonero breed have one monomer truncated at aa 496, a loss of the final 85 aa. SLICK3 expressed by the Limonero breed is truncated at aa 464, a loss of the final 115 aa. The truncated monomers are dominate in gene action and Mendelian inheritance. However, in one exemplary embodiment according to the invention, a break in the peptide anywhere after Y433 will result in the SLICK phenotype.

FIGS. 2A and 2B. FIG. 2A shows the genomic sequence of Exon 10 (see, GenBank AJ966356.4). The superscript numeral by the underlined residues identifies the following components of the sequence: ¹⁾ Start of Exon 10 (9^{th} exon); ²⁾ “tac” coding for tyrosine 433; ³⁾ first 3 residues in map shown in FIG. 3; ⁴) Residues modified to introduce Xbal site “tctaga” for SLICK1 RFLP identification; ⁵⁾ SLICK1 deletion of ‘c” results in frameshift; ⁶⁾ “t” to “a” introduces Nsil site “atgcat” for SLICK3 identification; ⁷⁾ SLICK3 “c” to “a” results in stop codon “taa”; ⁹⁾ residues modified to introduce Xbal1 site “tctaga” for SLICK2 RFLP identification; 10) Last 3 residues of FIG. 3. FIG. 2B is the amino acid sequence of the full length PRLR peptide. In this map, the residues underlined and identified by superscript are: ¹¹⁾ the extracellular domain (1-251); ¹²⁾ transmembrane domain; intracellular domain (295-581); ¹³) Y433; ¹⁴⁾ SLICK1 mutation results in break in protein synthesis; ¹⁵⁾ SLICK3 premature stop codon generated; ¹⁶⁾ SLICK2 premature stop codon generated.

FIG. 3 is a map of the PRLR gene at exon 10 illustrating the mutation strategy using TALENs.

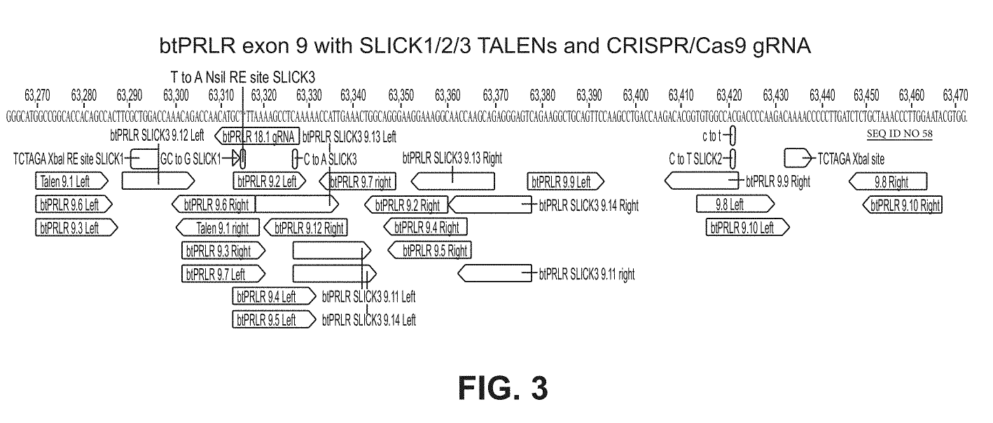

FIG. 4 are lysates of bovine cells introgressed for SLICK1 and showing restriction enzyme band patterns for Xbal digests. Left panel—clone mixtures, right panel—individual clones.

FIG. 5 cell lysates of bovine cells introgressed for SLICK2 showing cutting with Xbal restriction enzyme.

FIG. 6 is a gel showing banding pattern indicative of successful introgression of the SLICK2 mutation. RFLP=restriction fragment length polymorphism.

FIG. 7 gels showing cell lysates from bovine cells transfected with TALENs and oligo for SLICK3. Left panel, cell lysate; right panel, lysate of TALENs strategy 9.12 showing positive digestion with Nsil.

FIG. 8 RFLP analysis of individual clones transfected with TALENs and SLICK3 oligo.

### DETAILED DESCRIPTION OF THE EXEMPLARY EMBODIMENTS

Precision edited livestock animals and methods to provide them that express the slick phenotype are disclosed herein. The animals disclosed herein express a truncated allele for the prolactin receptor (PRLR) gene. When expressed, the livestock animals produce a PRLR that is missing up to the terminal 148 amino acids (aa) residues of the protein. In some embodiments the animal expresses a protein that is truncated by 147 or 146 aa. In some cases, the animal is missing the terminal 121 aa. In some embodiments, the Livestock animal expresses a PRLR that is missing the terminal 69 aa and exhibits the SLICK phenotype. Artisans will immediately appreciate that all ranges and values within the explicitly stated range are contemplated: e.g., from 148 to 69. That is, any PRLR expressing as its last amino acid tyrosine at position 433 of the protein translated from the mRNA having the GenBank Accession No. NM_001039726 will express the SLICK phenotype, with the caveat that truncation after tyrosine 512 may not express the SLICK phenotype.

Unless defined otherwise, all technical and scientific terms used herein have the same meanings as commonly understood by one of ordinary skill in the art to which this disclosure belongs. All publications and patents specifically mentioned herein are incorporated by reference for all purposes including describing and disclosing the chemicals, instruments, statistical analyses and methodologies which are reported in the publications which might be used in connection with the disclosure. All references cited in this specification are to be taken as indicative of the level of skill in the art. Nothing herein is to be construed as an admission that the disclosure is not entitled to antedate such disclosure by virtue of prior invention.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

“Additive Genetic Effects” as used herein means average individual gene effects that can be transmitted from parent to progeny.

“Allele” as used herein refers to an alternate form of a gene. It also can be thought of as variations of DNA sequence. For instance if an animal has the genotype for a specific gene of Bb, then both B and b are alleles.

As used herein, the term “breaking protein synthesis” refers to any deletion, insertion or mutation that creates a stop codon or frameshift that makes a premature stopping of protein synthesis.

“DNA Marker” refers to a specific DNA variation that can be tested for association with a physical characteristic.

“Genotype” refers to the genetic makeup of an animal.

“Genotyping (DNA marker testing)” refers to the process by which an animal is tested to determine the particular alleles it is carrying for a specific genetic test.

“Simple Traits” refers to traits such as coat color and horned status and some diseases that are carried by a single gene.

“Complex Traits” refers to traits such as reproduction, growth and carcass that are controlled by numerous genes.

“Complex allele”—coding region that has more than one mutation within it. This makes it more difficult to determine the effect of a given mutation because researchers cannot be sure which mutation within the allele is causing the effect.

“Copy number variation” (CNVs) a form of structural variation—are alterations of the DNA of a genome that results in the cell having an abnormal or, for certain genes, a normal variation in the number of copies of one or more sections of the DNA. CNVs correspond to relatively large regions of the genome that have been deleted (fewer than the normal number) or duplicated (more than the normal number) on certain chromosomes. For example, the chromosome that normally has sections in order as A-B-C-D might instead have sections A-B-C-“Repetitive element” patterns of nucleic acids (DNA or RNA) that occur in multiple copies throughout the genome. Repetitive DNA was first detected because of its rapid association kinetics.

“Quantitative variation” variation measured on a continuum (e.g. height in human beings) rather than in discrete units or categories. See continuous variation. The existence of a range of phenotypes for a specific character, differing by degree rather than by distinct qualitative differences.

“Homozygous” refers to having two copies of the same allele for a single gene such as BB.

“Heterozygous” refers to having different copies of alleles for a single gene such as Bb.”

“Locus” (plural “loci”) refers to the specific locations of a maker or a gene.

“Centimorgan (Cm)” a unit of recombinant frequency for measuring genetic linkage. It is defined as the distance between chromosome positions (also termed, loci or markers) for which the expected average number of intervening chromosomal crossovers in a single generation is 0.01. It is often used to infer distance along a chromosome. It is not a true physical distance however.

“Chromosomal crossover” (“crossing over”) is the exchange of genetic material between homologous chromosomes inherited by an individual from its mother and father. Each individual has a diploid set (two homologous chromosomes, e.g., 2n) one each inherited from its mother and father. During meiosis I the chromosomes duplicate (4n) and crossover between homologous regions of chromosomes received from the mother and father may occur resulting in new sets of genetic information within each chromosome. Meiosis I is followed by two phases of cell division resulting in four haploid (1n) gametes each carrying a unique set of genetic information. Because genetic recombination results in new gene sequences or combinations of genes, diversity is increased. Crossover usually occurs when homologous regions on homologous chromosomes break and then reconnect to the other chromosome.

“Marker Assisted Selection (MAS)” refers to the process by which DNA marker information is used to assist in making management decisions.

“Marker Panel” a combination of two or more DNA markers that are associated with a particular trait.

“Non-additive Genetic Effects” refers to effects such as dominance and epistasis. Codominance is the interaction of alleles at the same locus while epistasis is the interaction of alleles at different loci.

“Nucleotide” refers to a structural component of DNA that includes one of the four base chemicals: adenine (A), thymine (T), guanine (G), and cytosine (C).

“Phenotype” refers to the outward appearance of an animal that can be measured. Phenotypes are influenced by the genetic makeup of an animal and the environment.

“Single Nucleotide Polymorphism (SNP)” is a single nucleotide change in a DNA sequence.

“Haploid genotype” or “haplotype” refers to a combination of alleles, loci or DNA polymorphisms that are linked so as to cosegregate in a significant proportion of gametes during meiosis. The alleles of a haplotype may be in linkage disequilibrium (LD).

“Linkage disequilibrium (LD)” is the non-random association of alleles at different loci i.e. the presence of statistical associations between alleles at different loci that are different from what would be expected if alleles were independently, randomly sampled based on their individual allele frequencies. If there is no linkage disequilibrium between alleles at different loci they are said to be in linkage equilibrium.

The term “restriction fragment length polymorphism” or “RFLP” refers to any one of different DNA fragment lengths produced by restriction digestion of genomic DNA or cDNA with one or more endonuclease enzymes, wherein the fragment length varies between individuals in a population.

“Introgression” also known as “introgressive hybridization”, is the movement of a gene or allele (gene flow) from one species into the gene pool of another by the repeated backcrossing of an interspecific hybrid with one of its parent species. Purposeful introgression is a long-term process; it may take many hybrid generations before the backcrossing occurs.

“Nonmeiotic introgression” genetic introgression via introduction of a gene or allele in a diploid (non-gemetic) cell. Non-meiotic introgression does not rely on sexual reproduction and does not require backcrossing and, significantly, is carried out in a single generation. In non-meiotic introgression an allele is introduced into a haplotype via homologous recombination. The allele may be introduced at the site of an existing allele to be edited from the genome or the allele can be introduced at any other desirable site.

As used herein the term “genetic modification” refers to is the direct pu ation of an organism's genome using biotechnology.

As used herein the phrase “precision gene editing” means a process gene modification which allows geneticists to introduce (introgress) any natural trait into any breed, in a site specific manner without the use of recombinant DNA.

“Transcription activator-like effector nucleases (TALENs)” one technology for gene editing are artificial restriction enzymes generated by fusing a TAL effector DNA-binding domain to a DNA cleavage domain.

“Zinc finger nucleases (ZFNs)” as used herein are another technology useful for gene editing and are a class of engineered DNA-binding proteins that facilitate targeted editing of the genome by creating double-strand breaks in DNA at user-specified locations.

“Meganuclease” as used herein are another technology useful for gene editing and are endodeoxyribonucleases characterized by a large recognition site (double-stranded DNA sequences of 12 to 40 base pairs); as a result this site generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes.

“CRISPR/CAS” technology as used herein refers to “CRISPRs” (clustered regularly interspaced short palindromic repeats), segments of prokaryotic DNA containing short repetitions of base sequences. Each repetition is followed by short segments of “spacer DNA” from previous exposures to a bacterial virus or plasmid. “CAS” (CRISPR associated protein 9) is an RNA-guided DNA endonuclease enzyme associated with the CRISPR. By delivering the Cas9 protein and appropriate guide RNAs into a cell, the organism's genome can be cut at any desired location.

“Indel” as used herein is shorthand for “insertion” or “deletion” referring to a modification of the DNA in an organism.

As used herein the term “renucleated egg” refers to an enucleated egg used for somatic cell nuclear transfer in which the modified nucleus of a somatic cell has been introduced.

“Genetic marker” as used herein refers to a gene/allele or known DNA sequence with a known location on a chromosome. The markers may be any genetic marker e.g., one or more alleles, haplotypes, haplogroups, loci, quantitative trait loci, or DNA polymorphisms [restriction fragment length polymorphisms (RFLPs), amplified fragment length polymorphisms (AFLPs), single nuclear polymorphisms (SNPs), indels, short tandem repeats (STRs), microsatellites and minisatellites]. Conveniently, the markers are SNPs or STRs such as microsatellites, and more preferably SNPs. Preferably, the markers within each chromosome segment are in linkage disequilibrium.

As used herein the term “host animal” means an animal which has a native genetic complement of a recognized species or breed of animal.

As used herein, “native haplotype” or “native genome” means the natural DNA of a particular species or breed of animal that is chosen to be the recipient of a gene or allele that is not present in the host animal.

As used herein the term “target locus” means a specific location of a known allele on a chromosome.

As used herein, the term “quantitative trait” refers to a trait that fits into discrete categories. Quantitative traits occur as a continuous range of variation such as that amount of milk a particular breed can give or the length of a tail. Generally, a larger group of genes controls quantitative traits.

As used herein, the term “qualitative trait” is used to refer to a trait that falls into different categories. These categories do not have any certain order. As a general rule, qualitative traits are monogenic, meaning the trait is influenced by a single gene. Examples of qualitative traits include blood type and flower color, for example.

As used herein, the term “quantitative trait locus (QTL)” is a section of DNA (the locus) that correlates with variation in a phenotype (the quantitative trait).

As used herein the term “cloning” means production of genetically identical organisms asexually.

“Somatic cell nuclear transfer” (“SCNT”) is one strategy for cloning a viable embryo from a body cell and an egg cell. The technique consists of taking an enucleated oocyte (egg cell) and implanting a donor nucleus from a somatic (body) cell.

“Orthologous” as used herein refers to a gene with similar function to a gene in an evolutionarily related species. The identification of orthologues is useful for gene function prediction. In the case of livestock, orthologous genes are found throughout the animal kingdom and those found in other mammals may be particularly useful for transgenic replacement. This is particularly true for animals of the same species, breed or lineages wherein species are defined two animals so closely related as to being able to produce fertile offspring via sexual reproduction; breed is defined as a specific group of domestic animals having homogenous phenotype, homogenous behavior and other characteristics that define the animal from others of the same species; and wherein lineage is defined as continuous line of descent; a series of organisms, populations, cells, or genes connected by ancestor/descendent relationships. For example domesticated cattle are of two distinct lineages both arising from ancient aurochs. One lineage descends from the domestication of aurochs in the Middle East while the second distinct lineage descends from the domestication of the aurochs on the Indian subcontinent.

“Genotyping” or “genetic testing” generally refers to detecting one or more markers of interest e.g., SNPs in a sample from an individual being tested, and analyzing the results obtained to determine the haplotype of the subject. As will be apparent from the disclosure herein, it is one exemplary embodiment to detect the one or more markers of interest using a high-throughput system comprising a solid support consisting essentially of or having nucleic acids of different sequence bound directly or indirectly thereto, wherein each nucleic acid of different sequence comprises a polymorphic genetic marker derived from an ancestor or founder that is representative of the current population and, more preferably wherein said high-throughput system comprises sufficient markers to be representative of the genome of the current population. Preferred samples for genotyping comprise nucleic acid, e.g., RNA or genomic DNA and preferably genomic DNA. A breed of livestock animal can be readily established by evaluating its genetic markers.

“SLICK” as used herein refers to a phenotype of artiodactyls and cattle in particular which has a shortened coat length, hair density, hair type, sweat gland density and increased thermoregulatory efficiency. The gene effecting this phenotype has been identified as the prolactin receptor gene found on chromosome 20 of cattle.

The term “proximate” as used herein means close to.

Livestock may be genotyped to identify various genetic markers. Genotyping is a term that refers to the process of determining differences in the genetic make-up (genotype) of an individual by determining the individual's DNA sequence using a biological assay and comparing it to another individual's sequence or to a reference sequence. A genetic marker is a known DNA sequence, with a known location on a chromosome; they are consistently passed on through breeding, so they can be traced through a pedigree or phylogeny. Genetic markers can be a sequence comprising a plurality of bases, or a single nucleotide polymorphism (SNP) at a known location. The breed of a livestock animal can be readily established by evaluating its genetic markers. Many markers are known and there are many different measurement techniques that attempt to correlate the markers to traits of interest, or to establish a genetic value of an animal for purposes of future breeding or expected value.

### Homology Directed Repair (HDR)

Homology directed repair (HDR) is a mechanism in cells to repair ssDNA and double stranded DNA (dsDNA) lesions. This repair mechanism can be used by the cell when there is an HDR template present that has a sequence with significant homology to the lesion site. Specific binding, as that term is commonly used in the biological arts, refers to a molecule that binds to a target with a relatively high affinity compared to non-target tissues, and generally involves a plurality of non-covalent interactions, such as electrostatic interactions, van der Waals interactions, hydrogen bonding, and the like. Specific hybridization is a form of specific binding between nucleic acids that have complementary sequences. Proteins can also specifically bind to DNA, for instance, in TALENs or CRISPR/Cas9 systems or by Gal4 motifs. Introgression of an allele refers to a process of copying an exogenous allele over an endogenous allele with a template-guided process. The endogenous allele might actually be excised and replaced by an exogenous nucleic acid allele in some situations but present theory is that the process is a copying mechanism. Since alleles are gene pairs, there is significant homology between them. The allele might be a gene that encodes a protein, or it could have other functions such as encoding a bioactive RNA chain or providing a site for receiving a regulatory protein or RNA.

The HDR template is a nucleic acid that comprises the allele that is being introgressed. The template may be a dsDNA or a single-stranded DNA (ssDNA). ssDNA templates are preferably from about 20 to about 5000 residues although other lengths can be used. Artisans will immediately appreciate that all ranges and values within the explicitly stated range are contemplated; e.g., from 500 to 1500 residues, from 20 to 100 residues, and so forth. The template may further comprise flanking sequences that provide homology to DNA adjacent to the endogenous allele or the DNA that is to be replaced. The template may also comprise a sequence that is bound to a targeted nuclease system, and is thus the cognate binding site for the system's DNA-binding member. The term cognate refers to two biomolecules that typically interact, for example, a receptor and its ligand. In the context of HDR processes, one of the biomolecules may be designed with a sequence to bind with an intended, i.e., cognate, DNA site or protein site.

### Targeted Endonuclease Systems

Genome editing tools such as transcription activator-like effector nucleases (TALENs) and zinc finger nucleases (ZFNs) have impacted the fields of biotechnology, gene therapy and functional genomic studies in many organisms. More recently, RNA-guided endonucleases (RGENs) are directed to their target sites by a complementary RNA molecule. The Cas9/CRISPR system is a REGEN. tracrRNA is another such tool. These are examples of targeted nuclease systems: these system have a DNA-binding member that localizes the nuclease to a target site. The site is then cut by the nuclease. TALENs and ZFNs have the nuclease fused to the DNA-binding member. Cas9/CRISPR are cognates that find each other on the target DNA. The DNA-binding member has a cognate sequence in the chromosomal DNA. The DNA-binding member is typically designed in light of the intended cognate sequence so as to obtain a nucleolytic action at nor near an intended site. Certain embodiments are applicable to all such systems without limitation; including, embodiments that minimize nuclease re-cleavage, embodiments for making SNPs with precision at an intended residue, and placement of the allele that is being introgressed at the DNA-binding site.

### TALENs

The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA or a TALEN-pair.

The cipher for TALs has been reported (PCT Publication WO 2011/072246) wherein each DNA binding repeat is responsible for recognizing one base pair in the target DNA sequence. The residues may be assembled to target a DNA sequence. In brief, a target site for binding of a TALEN is determined and a fusion molecule comprising a nuclease and a series of RVDs that recognize the target site is created. Upon binding, the nuclease cleaves the DNA so that cellular repair machinery can operate to make a genetic modification at the cut ends. The term TALEN means a protein comprising a Transcription Activator-like (TAL) effector binding domain and a nuclease domain and includes monomeric TALENs that are functional per se as well as others that require dimerization with another monomeric TALEN. The dimerization can result in a homodimeric TALEN when both monomeric TALEN are identical or can result in a heterodimeric TALEN when monomeric TALEN are different. TALENs have been shown to induce gene modification in immortalized human cells by means of the two major eukaryotic DNA repair pathways, non-homologous end joining (NHEJ) and homology directed repair. TALENs are often used in pairs but monomeric TALENs are known. Cells for treatment by TALENs (and other genetic tools) include a cultured cell, an immortalized cell, a primary cell, a primary somatic cell, a zygote, a germ cell, a primordial germ cell, a blastocyst, or a stem cell. In some embodiments, a TAL effector can be used to target other protein domains (e.g., non-nuclease protein domains) to specific nucleotide sequences. For example, a TAL effector can be linked to a protein domain from, without limitation, a DNA 20 interacting enzyme (e.g., a methylase, a topoisomerase, an integrase, a transposase, or a ligase), a transcription activators or repressor, or a protein that interacts with or modifies other proteins such as histones. Applications of such TAL effector fusions include, for example, creating or modifying epigenetic regulatory elements, making site-specific insertions, deletions, or repairs in DNA, controlling gene expression, and modifying chromatin structure.

The term nuclease includes exonucleases and endonucleases. The term endonuclease refers to any wild-type or variant enzyme capable of catalyzing the hydrolysis (cleavage) of bonds between nucleic acids within a DNA or RNA molecule, preferably a DNA molecule. Non-limiting examples of endonucleases include type II restriction endonucleases such as FokI, HhaI, HindIII, NotI, BbvCI, EcoRI, BglII, and AlwI. Endonucleases comprise also rare-cutting endonucleases when having typically a polynucleotide recognition site of about 12-45 basepairs (bp) in length, more preferably of 14-45 bp. Rare-cutting endonucleases induce DNA double-strand breaks (DSBs) at a defined locus. Rare-cutting endonucleases can for example be a targeted endonuclease, a chimeric Zinc-Finger nuclease (ZFN) resulting from the fusion of engineered zinc-finger domains with the catalytic domain of a restriction enzyme such as FokI or a chemical endonuclease. In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences. Such chemical endonucleases are comprised in the term “endonuclease” according to the present invention. Examples of such endonuclease include I-See I, I-Chu L I-Cre I, I-Csm I, PI-See L PI-Tti L PI-Mtu I, I-Ceu I, I-See IL I-See III, HO, PI-Civ I, PI-Ctr L PI-Aae I, PI-Bsu I, PI-Dha I, PI-Dra L PI May L PI-Meh I, PI-Mfu L PI-Mfl I, PI-Mga L PI-Mgo I, PI-Min L PI-Mka L PI-Mle I, PI-Mma I, PI-30 Msh L PI-Msm I, PI-Mth I, PI-Mtu I, PI-Mxe I, PI-Npu I, PI-Pfu L PI-Rma I, PI-Spb I, PI-Ssp L PI-Fae L PI-Mja I, PI-Pho L PI-Tag L PI-Thy I, PI-Tko I, PI-Tsp I, I-MsoI.

A genetic modification made by TALENs or other tools may be, for example, chosen from the list consisting of an insertion, a deletion, insertion of an exogenous nucleic acid fragment, and a substitution. The term insertion is used broadly to mean either literal insertion into the chromosome or use of the exogenous sequence as a template for repair. In general, a target DNA site is identified and a TALEN-pair is created that will specifically bind to the site. The TALEN is delivered to the cell or embryo, e.g., as a protein, mRNA or by a vector that encodes the TALEN. The TALEN cleaves the DNA to make a double-strand break that is then repaired, often resulting in the creation of an indel, or incorporating sequences or polymorphisms contained in an accompanying exogenous nucleic acid that is either inserted into the chromosome or serves as a template for repair of the break with a modified sequence. This template-driven repair is a useful process for changing a chromosome, and provides for effective changes to cellular chromosomes.

The term exogenous nucleic acid means a nucleic acid that is added to the cell or embryo, regardless of whether the nucleic acid is the same or distinct from nucleic acid sequences naturally in the cell. The term nucleic acid fragment is broad and includes a chromosome, expression cassette, gene, DNA, RNA, mRNA, or portion thereof. The cell or embryo may be, for instance, chosen from the group consisting non-human vertebrates, non-human primates, cattle, horse, swine, sheep, chicken, avian, rabbit, goats, dog, cat, laboratory animal, and fish.

Some embodiments involve a composition or a method of making a genetically modified livestock and/or artiodactyl comprising introducing a TALEN-pair into livestock and/or an artiodactyl cell or embryo that makes a genetic modification to DNA of the cell or embryo at a site that is specifically bound by the TALEN-pair, and producing the livestock animal/artiodactyl from the cell. Direct injection may be used for the cell or embryo, e.g., into a zygote, blastocyst, or embryo. Alternatively, the TALEN and/or other factors may be introduced into a cell using any of many known techniques for introduction of proteins, RNA, mRNA, DNA, or vectors. Genetically modified animals may be made from the embryos or cells according to known processes, e.g., implantation of the embryo into a gestational host, or various cloning methods. The phrase “a genetic modification to DNA of the cell at a site that is specifically bound by the TALEN”, or the like, means that the genetic modification is made at the site cut by the nuclease on the TALEN when the TALEN is specifically bound to its target site. The nuclease does not cut exactly where the TALEN-pair binds, but rather at a defined site between the two binding sites.

Some embodiments involve a composition or a treatment of a cell that is used for cloning the animal. The cell may be a livestock and/or artiodactyl cell, a cultured cell, a primary cell, a primary somatic cell, a zygote, a germ cell, a primordial germ cell, or a stem cell. For example, an embodiment is a composition or a method of creating a genetic modification comprising exposing a plurality of primary cells in a culture to TALEN proteins or a nucleic acid encoding a TALEN or TALENs. The TALENs may be introduced as proteins or as nucleic acid fragments, e.g., encoded by mRNA or a DNA sequence in a vector.

### Zinc Finger Nucleases

Zinc-finger nucleases (ZFNs) are artificial restriction enzymes generated by fusing a zinc finger DNA-binding domain to a DNA-cleavage domain. Zinc finger domains can be engineered to target desired DNA sequences and this enables zinc-finger nucleases to target unique sequences within complex genomes. By taking advantage of endogenous DNA repair machinery, these reagents can be used to alter the genomes of higher organisms. ZFNs may be used in method of inactivating genes.

A zinc finger DNA-binding domain has about 30 amino acids and folds into a stable structure. Each finger primarily binds to a triplet within the DNA substrate. Amino acid residues at key positions contribute to most of the sequence-specific interactions with the DNA site. These amino acids can be changed while maintaining the remaining amino acids to preserve the necessary structure. Binding to longer DNA sequences is achieved by linking several domains in tandem. Other functionalities like non-specific FokI cleavage domain (N), transcription activator domains (A), transcription repressor domains (R) and methylases (M) can be fused to a ZFPs to form ZFNs respectively, zinc finger transcription activators (ZFA), zinc finger transcription repressors (ZFR, and zinc finger methylases (ZFM). Materials and methods for using zinc fingers and zinc finger nucleases for making genetically modified animals are disclosed in, e.g., U.S. Pat. No. 8,106,255; U.S. 2012/0192298; U.S. 2011/0023159; and U.S. 2011/0281306.

### Vectors and Nucleic Acids

A variety of nucleic acids may be introduced into cells, for knockout purposes, for inactivation of a gene, to obtain expression of a gene, or for other purposes. As used herein, the term nucleic acid includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained.

The target nucleic acid sequence can be operably linked to a regulatory region such as a promoter. Regulatory regions can be porcine regulatory regions or can be from other species. As used herein, operably linked refers to positioning of a regulatory region relative to a nucleic acid sequence in such a way as to permit or facilitate transcription of the target nucleic acid.

In general, type of promoter can be operably linked to a target nucleic acid sequence. Examples of promoters include, without limitation, tissue-specific promoters, constitutive promoters, inducible promoters, and promoters responsive or unresponsive to a particular stimulus. In some embodiments, a promoter that facilitates the expression of a nucleic acid molecule without significant tissue- or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter. See, for example, Xu et al., Hum. Gene Ther. 12:563, 2001; and Kiwaki et al., Hum. Gene Ther. 7:821, 1996.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable expressed markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban et al., Proc. Natl. Acad. Sci., 89:6861, 1992, for a review of Cre/lox technology, and Brand and Dymecki, Dev. Cell, 6:7, 2004. A transposon containing a Cre- or Flp-activatable transgene interrupted by a selectable marker gene also can be used to obtain transgenic animals with conditional expression of a transgene. For example, a promoter driving expression of the marker/transgene can be either ubiquitous or tissue-specific, which would result in the ubiquitous or tissue-specific expression of the marker in F0 animals (e.g., pigs). Tissue specific activation of the transgene can be accomplished, for example, by crossing a pig that ubiquitously expresses a marker-interrupted transgene to a pig expressing Cre or Flp in a tissue-specific manner, or by crossing a pig that expresses a marker-interrupted transgene in a tissue-specific manner to a pig that ubiquitously expresses Cre or Flp recombinase. Controlled expression of the transgene or controlled excision of the marker allows expression of the transgene.

In some embodiments, the exogenous nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and FLAG™ tag (Kodak, New Haven, Conn.).

Nucleic acid constructs can be introduced into embryonic, fetal, or adult artiodactyl/livestock cells of any type, including, for example, germ cells such as an oocyte or an egg, a progenitor cell, an adult or embryonic stem cell, a primordial germ cell, a kidney cell such as a PK-15 cell, an islet cell, a beta cell, a liver cell, or a fibroblast such as a dermal fibroblast, using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

In transposon systems, the transcriptional unit of a nucleic acid construct, i.e., the regulatory region operably linked to an exogenous nucleic acid sequence, is flanked by an inverted repeat of a transposon. Several transposon systems, including, for example, Sleeping Beauty (see, U.S. Pat. No. 6,613,752 and U.S. 2005/0003542); Frog Prince (Miskey et al., Nucleic Acids Res., 31:6873, 2003); Tol2 (Kawakami, Genome Biology, 8(Suppl.1):S7, 2007); Minos (Pavlopoulos et al., Genome Biology, 8(Suppl.1):52, 2007); Hsmarl (Miskey et al., Mol Cell Biol., 27:4589, 2007); and Passport have been developed to introduce nucleic acids into cells, including mice, human, and pig cells. The Sleeping Beauty transposon is particularly useful. A transposase can be delivered as a protein, encoded on the same nucleic acid construct as the exogenous nucleic acid, can be introduced on a separate nucleic acid construct, or provided as an mRNA (e.g., an in vitro-transcribed and capped mRNA).

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in animals have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, and also any necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggyBac).

As used herein, the term nucleic acid refers to both RNA and DNA, including, for example, cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand). The term transgenic is used broadly herein and refers to a genetically modified organism or genetically engineered organism whose genetic material has been altered using genetic engineering techniques. A knockout artiodactyl is thus transgenic regardless of whether or not exogenous genes or nucleic acids are expressed in the animal or its progeny.

### Genetically Modified Animals

Animals may be modified using TALENs or other genetic engineering tools, including recombinase fusion proteins, or various vectors that are known. A genetic modification made by such tools may comprise disruption of a gene. The term disruption of a gene refers to preventing the formation of a functional gene product. A gene product is functional only if it fulfills its normal (wild-type) functions. Disruption of the gene prevents expression of a functional factor encoded by the gene and comprises an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. Materials and methods of genetically modifying animals are further detailed in U.S. Pat. No. 8,518,701; U.S. 2010/0251395; and U.S. 2012/0222143 which are hereby incorporated herein by reference for all purposes; in case of conflict, the instant specification is controlling. The term trans-acting refers to processes acting on a target gene from a different molecule (i.e., intermolecular). A trans-acting element is usually a DNA sequence that contains a gene. This gene codes for a protein (or microRNA or other diffusible molecule) that is used in the regulation the target gene. The trans-acting gene may be on the same chromosome as the target gene, but the activity is via the intermediary protein or RNA that it encodes. Embodiments of trans-acting gene are, e.g., genes that encode targeting endonucleases. Inactivation of a gene using a dominant negative generally involves a trans-acting element. The term cis-regulatory or cis-acting means an action without coding for protein or RNA; in the context of gene inactivation, this generally means inactivation of the coding portion of a gene, or a promoter and/or operator that is necessary for expression of the functional gene.

Various techniques known in the art can be used to inactivate genes to make knock-out animals and/or to introduce nucleic acid constructs into animals to produce founder animals and to make animal lines, in which the knockout or nucleic acid construct is integrated into the genome. Such techniques include, without limitation, pronuclear microinjection (U.S. Pat. No. 4,873,191), retrovirus mediated gene transfer into germ lines (Van der Putten et al., Proc. Natl. Acad. Sci. USA, 82:6148-6152, 1985), gene targeting into embryonic stem cells (Thompson et al., Cell, 56:313-321, 1989), electroporation of embryos (Lo, Mol. Cell. Biol., 3:1803-1814, 1983), sperm-mediated gene transfer (Lavitrano et al., Proc. Natl. Acad. Sci. USA, 99:14230-14235, 2002; Lavitrano et al., Reprod. Fert. Develop., 18:19-23, 2006), and in vitro transformation of somatic cells, such as cumulus or mammary cells, or adult, fetal, or embryonic stem cells, followed by nuclear transplantation (Wilmut et al., Nature, 385:810-813, 1997; and Wakayama et al., Nature, 394:369-374, 1998). Pronuclear microinjection, sperm mediated gene transfer, and somatic cell nuclear transfer are particularly useful techniques. An animal that is genomically modified is an animal wherein all of its cells have the genetic modification, including its germ line cells. When methods are used that produce an animal that is mosaic in its genetic modification, the animals may be inbred and progeny that are genomically modified may be selected. Cloning, for instance, may be used to make a mosaic animal if its cells are modified at the blastocyst state, or genomic modification can take place when a single-cell is modified. Animals that are modified so they do not sexually mature can be homozygous or heterozygous for the modification, depending on the specific approach that is used. If a particular gene is inactivated by a knock out modification, homozygousity would normally be required. If a particular gene is inactivated by an RNA interference or dominant negative strategy, then heterozygosity is often adequate.

Typically, in pronuclear microinjection, a nucleic acid construct is introduced into a fertilized egg; 1 or 2 cell fertilized eggs are used as the pronuclei containing the genetic material from the sperm head and the egg are visible within the protoplasm. Pronuclear staged fertilized eggs can be obtained in vitro or in vivo (i.e., surgically recovered from the oviduct of donor animals). In vitro fertilized eggs can be produced as follows. For example, swine ovaries can be collected at an abattoir, and maintained at 22-28° C. during transport. Ovaries can be washed and isolated for follicular aspiration, and follicles ranging from 4-8 mm can be aspirated into 50 mL conical centrifuge tubes using 18 gauge needles and under vacuum. Follicular fluid and aspirated oocytes can be rinsed through pre-filters with commercial TL-HEPES (Minitube, Verona, Wis.). Oocytes surrounded by a compact cumulus mass can be selected and placed into TCM-199 OOCYTE MATURATION MEDIUM (Minitube, Verona, Wis.) supplemented with 0.1 mg/mL cysteine, 10 ng/mL epidermal growth factor, 10% porcine follicular fluid, 50 μM 2-mercaptoethanol, 0.5 mg/ml cAMP, 10 IU/mL each of pregnant mare serum gonadotropin (PMSG) and human chorionic gonadotropin (hCG) for approximately 22 hours in humidified air at 38.7° C. and 5% CO₂. Subsequently, the oocytes can be moved to fresh TCM-199 maturation medium, which will not contain cAMP, PMSG or hCG and incubated for an additional 22 hours. Matured oocytes can be stripped of their cumulus cells by vortexing in 0.1% hyaluronidase for 1 minute.

For swine, mature oocytes can be fertilized in 500 μl Minitube PORCPRO IVF MEDIUM SYSTEM (Minitube, Verona, Wis.) in Minitube 5-well fertilization dishes. In preparation for in vitro fertilization (IVF), freshly-collected or frozen boar semen can be washed and resuspended in PORCPRO IVF Medium to 4×10⁵ sperm. Sperm concentrations can be analyzed by computer assisted semen analysis (SPERMVISION, Minitube, Verona, Wis.). Final in vitro insemination can be performed in a 10 μl volume at a final concentration of approximately 40 motile sperm/oocyte, depending on boar. Incubate all fertilizing oocytes at 38.7° C. in 5.0% CO₂ atmosphere for 6 hours. Six hours post-insemination, presumptive zygotes can be washed twice in NCSU-23 and moved to 0.5 mL of the same medium. This system can produce 20-30% blastocysts routinely across most boars with a 10-30% polyspermic insemination rate.

Linearized nucleic acid constructs can be injected into one of the pronuclei. Then the injected eggs can be transferred to a recipient female (e.g., into the oviducts of a recipient female) and allowed to develop in the recipient female to produce the transgenic animals. In particular, in vitro fertilized embryos can be centrifuged at 15,000×g for 5 minutes to sediment lipids allowing visualization of the pronucleus. The embryos can be injected with using an Eppendorf FEMTOJET injector and can be cultured until blastocyst formation. Rates of embryo cleavage and blastocyst formation and quality can be recorded.

Embryos can be surgically transferred into uteri of asynchronous recipients. Typically, 100-200 (e.g., 150-200) embryos can be deposited into the ampulla-isthmus junction of the oviduct using a 5.5-inch TOMCAT® catheter. After surgery, real-time ultrasound examination of pregnancy can be performed.

In somatic cell nuclear transfer, a transgenic artiodactyl cell (e.g., a transgenic pig cell or bovine cell) such as an embryonic blastomere, fetal fibroblast, adult ear fibroblast, or granulosa cell that includes a nucleic acid construct described above, can be introduced into an enucleated oocyte to establish a combined cell. Oocytes can be enucleated by partial zona dissection near the polar body and then pressing out cytoplasm at the dissection area. Typically, an injection pipette with a sharp beveled tip is used to inject the transgenic cell into an enucleated oocyte arrested at meiosis 2. In some conventions, oocytes arrested at meiosis-2 are termed eggs. After producing a porcine or bovine embryo (e.g., by fusing and activating the oocyte), the embryo is transferred to the oviducts of a recipient female, about 20 to 24 hours after activation. See, for example, Cibelli et al., Science, 280:1256-1258, 1998; and U.S. Pat. No. 6,548,741. For pigs, recipient females can be checked for pregnancy approximately 20-21 days after transfer of the embryos.

Standard breeding techniques can be used to create animals that are homozygous for the exogenous nucleic acid from the initial heterozygous founder animals. Homozygosity may not be required, however. Transgenic pigs described herein can be bred with other pigs of interest.

In some embodiments, a nucleic acid of interest and a selectable marker can be provided on separate transposons and provided to either embryos or cells in unequal amount, where the amount of transposon containing the selectable marker far exceeds (5-10 fold excess) the transposon containing the nucleic acid of interest. Transgenic cells or animals expressing the nucleic acid of interest can be isolated based on presence and expression of the selectable marker. Because the transposons will integrate into the genome in a precise and unlinked way (independent transposition events), the nucleic acid of interest and the selectable marker are not genetically linked and can easily be separated by genetic segregation through standard breeding. Thus, transgenic animals can be produced that are not constrained to retain selectable markers in subsequent generations, an issue of some concern from a public safety perspective.

Once transgenic animals have been generated, expression of an exogenous nucleic acid can be assessed using standard techniques. Initial screening can be accomplished by Southern blot analysis to determine whether or not integration of the construct has taken place. For a description of Southern analysis, see sections 9.37-9.52 of Sambrook et al., Molecular Cloning, A Laboratory Manual, second edition, Cold Spring Harbor Press, Plainview; NY., 1989. Polymerase chain reaction (PCR) techniques also can be used in the initial screening. PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. PCR is described in, for example PCR Primer: A Laboratory Manual, ed. Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplified. See, for example, Lewis, Genetic Engineering News, 12:1, 1992; Guatelli et al., Proc. Natl. Acad. Sci. USA, 87:1874, 1990; and Weiss, Science, 254:1292, 1991. At the blastocyst stage, embryos can be individually processed for analysis by PCR, Southern hybridization and splinkerette PCR (see, e.g., Dupuy et al., Proc Natl Acad Sci USA, 99:4495, 2002).

Expression of a nucleic acid sequence encoding a polypeptide in the tissues of transgenic pigs can be assessed using techniques that include, for example, Northern blot analysis of tissue samples obtained from the animal, in situ hybridization analysis, Western analysis, immunoassays such as enzyme-linked immunosorbent assays, and reverse-transcriptase PCR (RT-PCR).

### Interfering RNAs

A variety of interfering RNA (RNAi) are known. Double-stranded RNA (dsRNA) induces sequence-specific degradation of homologous gene transcripts. RNA-induced silencing complex (RISC) metabolizes dsRNA to small 21-23-nucleotide small interfering RNAs (siRNAs). RISC contains a double stranded RNAse (dsRNase, e.g., Dicer) and ssRNase (e.g., Argonaut 2 or Ago2). RISC utilizes antisense strand as a guide to find a cleavable target. Both siRNAs and microRNAs (miRNAs) are known. A method of disrupting a gene in a genetically modified animal comprises inducing RNA interference against a target gene and/or nucleic acid such that expression of the target gene and/or nucleic acid is reduced.

For example the exogenous nucleic acid sequence can induce RNA interference against a nucleic acid encoding a polypeptide. For example, double-stranded small interfering RNA (siRNA) or small hairpin RNA (shRNA) homologous to a target DNA can be used to reduce expression of that DNA. Constructs for siRNA can be produced as described, for example, in Fire et al., Nature, 391:806, 1998; Romano and Masino, Mol. Microbiol., 6:3343, 1992; Cogoni et al., EMBO J., 15:3153, 1996; Cogoni and Masino, Nature, 399:166, 1999; Misquitta and Paterson Proc. Natl. Acad. Sci. USA, 96:1451, 1999; and Kennerdell and Carthew, Cell, 95:1017, 1998. Constructs for shRNA can be produced as described by McIntyre and Fanning (2006) BMC Biotechnology 6:1. In general, shRNAs are transcribed as a single-stranded RNA molecule containing complementary regions, which can anneal and form short hairpins.

The probability of finding a single, individual functional siRNA or miRNA directed to a specific gene is high. The predictability of a specific sequence of siRNA, for instance, is about 50% but a number of interfering RNAs may be made with good confidence that at least one of them will be effective.

Embodiments include an in vitro cell, an in vivo cell, and a genetically modified animal such as a livestock animal that express an RNAi directed against a gene, e.g., a gene selective for a developmental stage. The RNAi may be, for instance, selected from the group consisting of siRNA, shRNA, dsRNA, RISC and miRNA.

### Inducible Systems

An inducible system may be used to control expression of a gene. Various inducible systems are known that allow spatiotemporal control of expression of a gene. Several have been proven to be functional in vivo in transgenic animals. The term inducible system includes traditional promoters and inducible gene expression elements.

An example of an inducible system is the tetracycline (tet)-on promoter system, which can be used to regulate transcription of the nucleic acid. In this system, a mutated Tet repressor (TetR) is fused to the activation domain of herpes simplex virus VP16 trans-activator protein to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent.

The tetracycline-inducible system and the Cre/loxP recombinase system (either constitutive or inducible) are among the more commonly used inducible systems. The tetracycline-inducible system involves a tetracycline-controlled transactivator (tTA)/reverse tTA (rtTA). A method to use these systems in vivo involves generating two lines of genetically modified animals. One animal line expresses the activator (tTA, rtTA, or Cre recombinase) under the control of a selected promoter. Another set of transgenic animals express the acceptor, in which the expression of the gene of interest (or the gene to be modified) is under the control of the target sequence for the tTA/rtTA transactivators (or is flanked by loxP sequences). Mating the two strains of mice provides control of gene expression.

The tetracycline-dependent regulatory systems (tet systems) rely on two components, i.e., a tetracycline-controlled transactivator (tTA or rtTA) and a tTA/rtTA-dependent promoter that controls expression of a downstream cDNA, in a tetracycline-dependent manner. In the absence of tetracycline or its derivatives (such as doxycycline), tTA binds to tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. Administration of tetracycline or its derivatives allows temporal control of transgene expression in vivo. rtTA is a variant of tTA that is not functional in the absence of doxycycline but requires the presence of the ligand for transactivation. This tet system is therefore termed tet-ON. The tet systems have been used in vivo for the inducible expression of several transgenes, encoding, e.g., reporter genes, oncogenes, or proteins involved in a signaling cascade.

The Cre/lox system uses the Cre recombinase, which catalyzes site-specific recombination by crossover between two distant Cre recognition sequences, i.e., loxP sites. A DNA sequence introduced between the two loxP sequences (termed floxed DNA) is excised by Cre-mediated recombination. Control of Cre expression in a transgenic animal, using either spatial control (with a tissue- or cell-specific promoter) or temporal control (with an inducible system), results in control of DNA excision between the two loxP sites. One application is for conditional gene inactivation (conditional knockout). Another approach is for protein over-expression, wherein a foxed stop codon is inserted between the promoter sequence and the DNA of interest. Genetically modified animals do not express the transgene until Cre is expressed, leading to excision of the floxed stop codon. This system has been applied to tissue-specific oncogenesis and controlled antigene receptor expression in B lymphocytes. Inducible Cre recombinases have also been developed. The inducible Cre recombinase is activated only by administration of an exogenous ligand. The inducible Cre recombinases are fusion proteins containing the original Cre recombinase and a specific ligand-binding domain. The functional activity of the Cre recombinase is dependent on an external ligand that is able to bind to this specific domain in the fusion protein.

Embodiments include an in vitro cell, an in vivo cell, and a genetically modified animal such as a livestock animal that comprise a gene under control of an inducible system. The genetic modification of an animal may be genomic or mosaic. The inducible system may be, for instance, selected from the group consisting of Tet-On, Tet-Off, Cre-lox, and Hifl alpha. An embodiment is a gene set forth herein.

### Dominant Negatives

Genes may thus be disrupted not only by removal or RNAi suppression but also by creation/expression of a dominant negative variant of a protein which has inhibitory effects on the normal function of that gene product. The expression of a dominant negative (DN) gene can result in an altered phenotype, exerted by a) a titration effect; the DN PASSIVELY competes with an endogenous gene product for either a cooperative factor or the normal target of the endogenous gene without elaborating the same activity, b) a poison pill (or monkey wrench) effect wherein the dominant negative gene product ACTIVELY interferes with a process required for normal gene function, c) a feedback effect, wherein the DN ACTIVELY stimulates a negative regulator of the gene function.

### Founder Animals, Animal Lines, Traits, and Reproduction

Founder animals (F0 generation) may be produced by cloning and other methods described herein. The founders can be homozygous for a genetic modification, as in the case where a zygote or a primary cell undergoes a homozygous modification. Similarly, founders can also be made that are heterozygous. The founders may be genomically modified, meaning that the cells in their genome have undergone modification. Founders can be mosaic for a modification, as may happen when vectors are introduced into one of a plurality of cells in an embryo, typically at a blastocyst stage. Progeny of mosaic animals may be tested to identify progeny that are genomically modified. An animal line is established when a pool of animals has been created that can be reproduced sexually or by assisted reproductive techniques, with heterogeneous or homozygous progeny consistently expressing the modification.

In livestock, many alleles are known to be linked to various traits such as production traits, type traits, workability traits, and other functional traits. Artisans are accustomed to monitoring and quantifying these traits, e.g., Visscher et al., Livestock Production Science, 40:123-137, 1994; U.S. Pat. No. 7,709,206; U.S. 2001/0016315; U.S. 2011/0023140; and U.S. 2005/0153317. An animal line may include a trait chosen from a trait in the group consisting of a production trait, a type trait, a workability trait, a fertility trait, a mothering trait, and a disease resistance trait. Further traits include expression of a recombinant gene product.

### Recombinases

Embodiments of the invention include administration of a targeted nuclease system with a recombinase (e.g., a RecA protein, a Rad51) or other DNA-binding protein associated with DNA recombination. A recombinase forms a filament with a nucleic acid fragment and, in effect, searches cellular DNA to find a DNA sequence substantially homologous to the sequence. For instance a recombinase may be combined with a nucleic acid sequence that serves as a template for HDR. The recombinase is then combined with the HDR template to form a filament and placed into the cell. The recombinase and/or HDR template that combines with the recombinase may be placed in the cell or embryo as a protein, an mRNA, or with a vector that encodes the recombinase. The disclosure of U.S. 2011/0059160 (U.S. patent application Ser. No. 12/869,232) is hereby incorporated herein by reference for all purposes; in case of conflict, the specification is controlling. The term recombinase refers to a genetic recombination enzyme that enzymatically catalyzes, in a cell, the joining of relatively short pieces of DNA between two relatively longer DNA strands. Recombinases include Cre recombinase, Hin recombinase, RecA, RAD51, Cre, and FLP. Cre recombinase is a Type I topoisomerase from P1 bacteriophage that catalyzes site-specific recombination of DNA between loxP sites. Hin recombinase is a 21 kD protein composed of 198 amino acids that is found in the bacteria Salmonella. Hin belongs to the serine recombinase family of DNA invertases in which it relies on the active site serine to initiate DNA cleavage and recombination. RAD51 is a human gene. The protein encoded by this gene is a member of the RAD51 protein family which assists in repair of DNA double strand breaks. RAD51 family members are homologous to the bacterial RecA and yeast Rad51. Cre recombinase is an enzyme that is used in experiments to delete specific sequences that are flanked by loxP sites. FLP refers to Flippase recombination enzyme (FLP or Flp) derived from the 2μ plasmid of the baker's yeast Saccharomyces cerevisiae.

Herein, “RecA” or “RecA protein” refers to a family of RecA-like recombination proteins having essentially all or most of the same functions, particularly: (i) the ability to position properly oligonucleotides or polynucleotides on their homologous targets for subsequent extension by DNA polymerases; (ii) the ability topologically to prepare duplex nucleic acid for DNA synthesis; and, (iii) the ability of RecA/oligonucleotide or RecA/polynucleotide complexes efficiently to find and bind to complementary sequences. The best characterized RecA protein is from E. coli; in addition to the original allelic form of the protein a number of mutant RecA-like proteins have been identified, for example, RecA803. Further, many organisms have RecA-like strand-transfer proteins including, for example, yeast, Drosophila, mammals including humans, and plants. These proteins include, for example, Rec1, Rec2, Rad51, Rad51B, Rad51C, Rad51D, Rad51E, XRCC2 and DMC1. An embodiment of the recombination protein is the RecA protein of E. coli. Alternatively, the RecA protein can be the mutant RecA-803 protein of E. coli, a RecA protein from another bacterial source or a homologous recombination protein from another organism.

### Compositions and Kits

The present invention also provides compositions and kits containing, for example, nucleic acid molecules encoding site-specific endonucleases, CRISPR, Cas9, ZNFs, TALENs, RecA-gal4 fusions, polypeptides of the same, compositions containing such nucleic acid molecules or polypeptides, or engineered cell lines. An HDR may also be provided that is effective for introgression of an indicated allele. Such items can be used, for example, as research tools, or therapeutically.

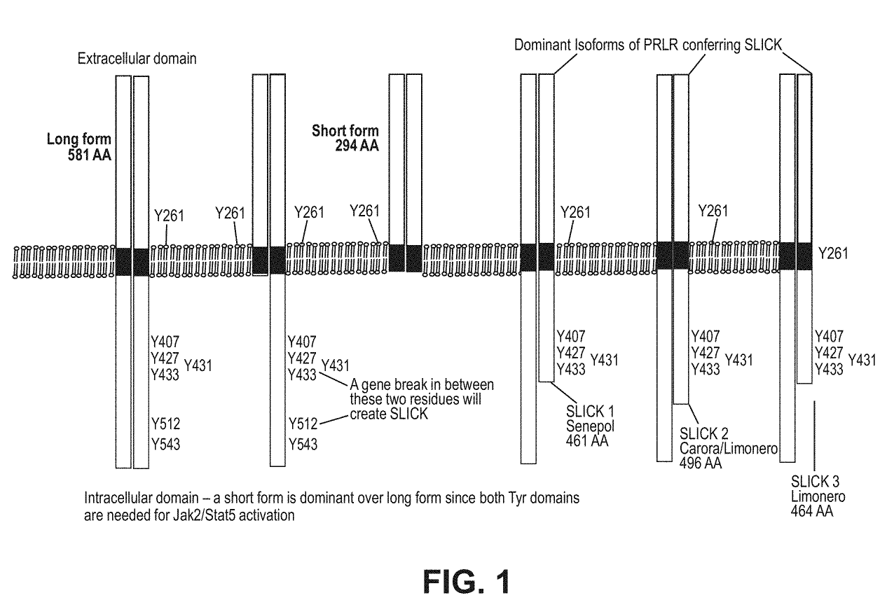

The phenotype for SLICK was clearly a qualitative trait showing monogenic inheritance. Cross breeding of cattle to take advantage of the SLICK phenotype also showed that the trait was dominant, showing expression in heterozygous animals. Several groups have recently tried to isolate the gene and Littlejohn et al (Nat Commun 5: 5861 (2014) identified a single base deletion in exon 10 (exons counted from exon 2 resulting in the 9^{th} exon being termed exon 10) in senepol cattle resulting in a frameshift, introducing a premature stop codon resulting in a peptide of 461AA due to a loss of the terminal 120 aa of the WT peptide. See, FIG. 1.

The gene for the prolactin receptor is found on chromosome 20 of cattle (Bos Taurus) and has nine exons and codes for a protein of 581 amino acids in length. Each monomer has an extracellular domain, transmembrane domain and an intracellular domain and dimerizes as shown in FIG. 1 to form a functional receptor. There are several isoforms of PRLR including one that has no intracellular domain. However, the 294AA short from is not expressed in bovine animals and may be tissue specific always being expressed with the long form of the protein.

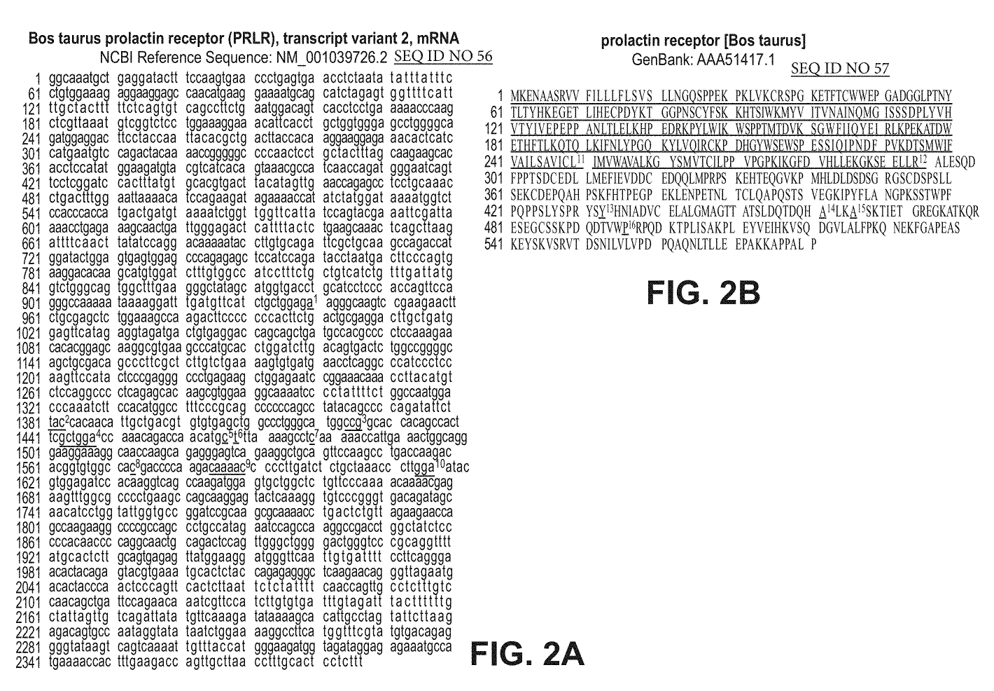

Other breeds of cattle also express SLICK phenotypes and investigators have recently isolated two other isoforms of the PRLR gene that result in truncated PRLR peptides. SLICK2 (as coined herein) is expressed by Carora/Limonero cattle and is a single base mutation resulting in a premature stop codon resulting in a peptide of 496AA. SLICK3 is expressed by Limonero cattle and is a single base mutation resulting in a protein truncated at 464AA. See, FIG. 1 and FIG. 2A showing the nucleotide sequence of PRLR mRNA as identified by GenBank Accession No. NM_001039726. Shown at residue 940 is the start of exon 10 while the coding site for tyrosine 433 is coded for by residues “tac” at 1381 to 1383. The mutation leading to SLICK1 is a deletion of “c” at 1466; SLICK3 is “c” at 1478 and the mutation giving rise to SLICK2 is a mutation of the “c” at 1573. The amino acids and their position in the peptide are illustrated in FIG. 2B.

The PRLR undergoes tyrosine phosphorylation after stimulation by PRL in which JAK2 phosphorylates multiple tyrosine sites in the PRLR cytoplasmic loop and loop-associated STAT5a and STAT5b. Subsequently tyrosine phosphorylated STAT5 dissociates from the loop and forms an active dimer and translates to the nucleus regulating gene functions associated with PRL. Thus, tyrosine residues are thought to be highly functional for PRLR signaling. Therefore, without being held to any specific theory, the present inventors hypothesize that, due to the functionality of tyrosine, because tyrosine Y261 is present regardless of coat phenotype and because SLICK is evident at least by truncation of PRLR after AA 461 that truncation of PRLR up to the preceding tyrosine Y433 will result in a SLICK phenotype.

As disclosed herein are provided livestock animals, in one embodiment artiodacyls and cattle especially, which express the slick phenotype by being modified genetically to express a PRLR gene which has a break in synthesis of the PRLR peptide due to a mutation encoding an insert, deletion, premature stop codon or other modification resulting in a PRLR peptide that is lacking up to 148 terminal amino acids. In various exemplary embodiments, modification of the PRLR gene is achieved by nonmeiotic introgression of the PRLR gene using right and left Transcription activator-like effector nucleases (TALENs) constructs and appropriate homology directed repair (HDR) templates to introduce mutations resulting a break in protein synthesis in the PRLR at some point in the peptide after the tyrosin residue at position 433 as identified in the peptide sequence having the GenBank Accession No. AAA51417. In some embodiments the break in protein synthesis is before the tyrosine residue at 512 of the peptide. The use of nonmeiotic introgression is known in the art and is described at length in U.S. Published Patent Applications 2012/0222143; 2013/0117870 and 2015/0067898 hereby incorporated by reference in their entirety for all purposes.

Various exemplary embodiments of devices and compounds as generally described above and methods according to this invention, will be understood more readily by reference to the following examples, which are provided by way of illustration and are not intended to be limiting of the invention in any fashion.

### Example 1

### Talens Design and Production

TALEN designing and production. Candidate TALEN target DNA sequences and RVD sequences were identified using the online tool “TAL Effector Nucleotide Targeter” (tale-nt.cac.cornell.edu/about). Plasmids for TALEN DNA transfection or in vitro TALEN mRNA transcription were then constructed by following the Golden Gate Assembly protocol using pC-GoldyTALEN (Addgene ID 38143) and RClscript-GoldyTALEN (Addgene ID 38143) as final destination vectors (2). The final pC-GoldyTALEN vectors were prepared by using PureLink® HiPure Plasmid Midiprep Kit (Life Technologies) and sequenced before usage. Assembled RClscript vectors prepared using the QIAprep Spin Miniprep kit (Qiagen) were linearized by SacI to be used as templates for in vitro TALEN mRNA transcription using the mMESSAGE mMACHINE® T3 Kit (Ambion) as indicated previously. Modified mRNA was synthesized from RCIScript-GoldyTALEN vectors as previously described substituting a ribonucleotide cocktail consisting of 3′-O-Me-m7G(5′)ppp(5′)G RNA cap analog (New England Biolabs), 5-methylcytidine triphosphate pseudouridine triphosphate (TriLink Biotechnologies, San Diego, Calif.) and adenosine triphosphate and guanosine triphosphate. Final nucleotide reaction concentrations are 6 mM for the cap analog, 1.5 mM for guanosine triphosphate, and 7.5 mM for the other nucleotides. Resulting mRNA was DNAse treated prior to purification using the MEGAclear Reaction Cleanup kit (Applied Biosciences). Table I provides a list of RVD sequences used.

TABLE I
TALEN and CRISPR/Cas9 target sequences.

| Talen Pair | Talen RVD sequence Left Arm | DNA Target sequence (Sense strand) |
|---|---|---|
| btSLICK1 | NN NN HD HD NN NN HD NI HD HD NI HD | GGCCGGCACCACAGCCACTTCGCTGGACCAAACAGACCAACATGCTTTA |
| 9.1 | NI NN HD HD | Seq ID 29 |
| Seq ID | NG NI NI NI NN HD NI NG NN NG NG NN | |
| 1/2 | NN NG HD NG NN NG | |
| btSLICK1 | NN HD NG NG NG NI NI NI NI NN HD HD | GCTTTAAAAGCCTCAAAAACCATTGAAACTGGCAGGGAAGGAAAGGC |
| 9.2 | NG HD NI NI | Seq ID 30 |
| Seq ID | NN HD HD NG NG NG HD HD NG NG HD HD | |
| 3/4 | HD NG NN HD HD NI | |
| btSLICK1 | NN NN HD HD NN NN HD NI HD HD NI HD | GGCCGGCACCACAGCCACTTCGCTGGACCAAACAGACCAACATGCTTTAA |
| 9.3 | NI NN HD HD NI HD | Seq ID 31 |
| Seq ID | NG NG NI NI NI NN HD NI NG NN NG NG | |
| 5/6 | NN NN NG HD NG NN | |
| btSLICK1 | NN HD NG NG NG NI NI NI NI NN HD HD | GCTTTAAAAGCCTCAAAAACCATTGAAACTGGCAGGGAAGGAAAGGCAACC |
| 9.4 | NG HD NI NI NI NI | Seq ID 32 |
| Seq ID | NN NN NG NG NN HD HD NG NG NG HD HD | |
| 7/8 | NG NG HD HD HD NG | |
| btSLICK1 | NN HD NG NG NG NI NI NI NI NN HD HD | GCTTTAAAAGCCTCAAAAACCATTGAAACTGGCAGGGAAGGAAAGGCAACCA |
| 9.5 | NG HD NI NI NI NI | Seq ID 33 |
| Seq ID | NG NN NN NG NG NN HD HD NG NG NG HD | |
| 9/10 | HD NG NG HD HD HD | |
| btSLICK1 | NN NN HD HD NN NN HD NI HD HD NI HD | GGCCGGCACCACAGCCACTTCGCTGGACCAAACAGACCAACATGCTTT |
| 9.6 | NI NN HD HD NI | Seq ID 34 |
| Seq ID | NI NI NI NN HD NI NG NN NG NG NN NN | |
| 11/12 | NG HD NG NN NG NG | |
| btSLICK1 | HD NI NN NI HD HD NI NI HD NI NG NN | CAGACCAACATGCTTTAAAAGCCTCAAAAACCATTGAAACTGGCAG |
| 9.7 | HD NG NG NG NI NI | Seq ID 35 |
| Seq ID | HD HD NI NG NG NN NI NI NI HD NG NN | |
| 13/14 | NN HD NI NN | |
| btSLICK2 | NN NG NN NN HD HD NI HD NN NI HD HD | GTGGCCACGACCCCAAGACAAAACCCCCTTGATCTCTGCTAAACCCTTGG |
| 9.8 | HD HD NI NI NN | Seq ID 36 |
| Seq ID | HD HD NI NI NN NN NN NG NG NG NI NN | |
| 15/16 | HD NI NN NI NN | |
| btSLICK2 | HD NI NN NI NI NN NN HD NG NN HD NI | CAGAAGGCTGCAGTTCCAAGCCTGACCAAGACACGGTGTGGCCACG |
| 9.9 | NN NG NG HD HD | Seq ID 37 |
| Seq ID | HD NN NG NN NN HD HD NI HD NI HD HD | |
| 17/18 | NN NG NN NG | |
| btSLICK2 | NN NN HD HD NI HD NN NI HD HD HD HD | GGCCACGACCCCAAGACAAAACCCCCTTGATCTCTGCTAAACCCTTGGAAT |
| 9.10 | NI NI NN NI HD NI | Seq ID 38 |
| Seq ID | NI NG NG HD HD NI NI NN NN NN NG NG | |
| 19/20 | NG NI NN HD NI | |
| btSLICK3 | HD NI NI NI NI NI HD HD NI NG NG NN | CAAAAACCATTGAAACTGGCAGGGAAGGAAAGGCAACCAAGCAGAGGGAGTC |
| 9.11 | NI NI NI HD NG | Seq ID 39 |
| Seq ID | NN NI HD NG HD HD HD NG HD NG NN HD | |
| 21/22 | NG NG NN NN | |
| btSLICK3 | NG HD NN HD NG NN NN NI HD HD NI NI | TCGCTGGACCAAACAGACCAACATGCTTTAAAAGCCTCAAAAACCATTG |
| 9.12 | NI HD NI NN | Seq ID 40 |
| Seq ID | HD NI NI NG NN NN NG NG NG NG NG NN | |
| 23/24 | NI NN NN HD NG NG | |
| btSLICK3 | NI NI NI NI NN HD HD NG HD NI NI NI | AAAAGCCTCAAAAACCATTGAAACTGGCAGGGAAGGAAAGGCAACCAAGCAG |
| 9.13 | NI NI HD HD NI NG | Seq ID 41 |
| Seq ID | HD NG NN HD NG NG NN NN NG NG NN HD | |
| 25/26 | HD NG NG NG HD HD | |
| btSLICK3 | HD NI NI NI NI NI HD HD NI NG NG NN | CAAAAACCATTGAAACTGGCAGGGAAGGAAAGGCAACCAAGCAGAGGGAGTC |
| 9.14 | NI NI NI HD NG NN | Seq ID 42 |
| Seq ID | NN NI HD NG HD HD HD NG HD NG NN HD | |
| 27/28 | NG NG NN NN NG NG | |
| btSLICK1 | | GAGGCTTTTAAAGCATGT (reverse strand) |
| 18.1 | | Seq ID 43 |
| sgRNA | | |

Note:
RVD sequences for left and right TALEN monomers are shown top and bottom respectively oriented from the N to C terminus.
Bold text indicates TALEN binding sites.

### Oligonucleotide Templates

All oligonucleotide templates were synthesized by Integrated DNA Technologies, 100 nmole synthesis purified by standard desalting, and resuspended to 400 μM in TE. See, Table II for the list of oligo templates.

TABLE II
Introgression templates

| | ssODN | | Sequence |
|---|---|---|---|
| Talen Pair | design | Sequence | ID # |
| btSLICK1 | SLICK1_ | ggccctgggcatggccggcaccacagccacttctctagaccaaacagaccaaca | Seq ID 44 |
| 9.1 | XbaI | tg[DelC]tttaaaagcctcaaaaaccattgaaactggcagg | |
| btSLICK1 | SLICK1_ | Ggccctgggcatggccggcaccacagccacttcgctggaccaaacagaccaac | Seq ID 45 |
| 9.1 | native | atgctttaaaagcctcaaaaaccattgaaactggcagg | |
| btSLICK2 | SLICK2_ | agcctgaccaagacacggtgtggccaTgaccccaagactctagaccatgatct | Seq ID 46 |
| 9.8 | XbaI | ctgctaaaccatggaatacgtggagatccacaagg | |
| btSLICK2 | SLICK2_ | Agcctgaccaagacacggtgtggccacgaccccaagacaaaacccccttgatct | Seq ID 47 |
| 9.8 | five | ctgctaaacccttggaatacgtggagatccacaagg | |
| btSLICK3 | SLICK3_ | GcaccacagccacttcgctggaccaaacagaccaacatgcattaaaagcctAaa | Seq ID 48 |
| 9.12 | NsiI | aaaccattgaaactggcagggaaggaaaggcaacca | |
| btSLICK3 | SLICK3_ | Gcaccacagccacttcgctggaccaaacagaccaacatgattaaaagcctcaaa | Seq ID 49 |
| 9.12 | native | aaccattgaaactggcagggaaggaaaggcaacca | |

Capitalized text represents intended SNPs; bold text indicates nucleotide changes to generate restriction sites for RFLP screening, double underline text indicates TALEN sites; novel restriction sites are underlined. [DelC] indicates deletion of the cytosine nucleotide at this position. Native notation indicates the template that will only introduce the native SLICK1, 2 or 3 mutation with no additional base changes.

### Example 2

### Tissue Culture and Transfection.

Bovine fibroblasts were maintained at 37 or 30° C. (as indicated) at 5% CO2 in DMEM supplemented with 10% fetal bovine serum, 100 I.U./ml penicillin and streptomycin, and 2 mM L-Glutamine. For transfection, all TALENs, CRISPR/Cas9 and HDR templates were delivered through transfection using the Neon Transfection system (Life Technologies) unless otherwise stated. Briefly, low passage bovine fibroblasts reaching 100% confluence were split 1:2 and harvested the next day at 70-80% confluence. Each transfection was comprised of 500,000-600,000 cells resuspended in buffer “R” mixed with mRNA and oligos and electroporated using the 100 ul tips by the following parameters: input Voltage; 1800V; Pulse Width; 20 ms; and Pulse Number; 1. Typically, 0.1-5 of TALEN mRNA and 2-5 μM of oligos specific for the SLICK mutation desired were included in each transfection along with oligos entering the required restriction site for RFLP analysis. After transfection, cells were divided 60:40 into two separate wells of a 6-well dish for three days' culture at either 30 or 37° C. respectively. After three days, cell populations were expanded and at 37° C. until at least day 10 to assess stability of edits. Table III provides a summary of positively transfected cells from each treatment group.

### Dilution Cloning:

Three days post transfection, 50 to 250 cells were seeded onto 10 cm dishes and cultured until individual colonies reached circa 5 mm in diameter. At this point, 6 ml of TrypLE (Life Technologies) 1:5 (vol/vol) diluted in PBS was added and colonies were aspirated, transferred into wells of a 24-well dish well and cultured under the same conditions. Colonies reaching confluence were collected and divided for cryopreservation and genotyping.

TABLE III

| Talen Name | % Cell |
|---|---|
| btPRLR 9.1 (SLICK1) | 20.9 |
| btPRLR 9.2 (SLICK1) | 0 |
| btPRLR 9.3 (SLICK1) | 0 |
| btPRLR 9.4 (SLICK1) | 0 |
| btPRLR 9.5 (SLICK1) | 13.9 |
| btPRLR 9.6 (SLICK1) | 0 |
| btPRLR 9.7 (SLICK1) | 0 |
| btPRLR 18.1 gRNA (SLICK 1) | 0 |
| btPRLR 9.8 (SLICK2) | 10.1 |
| btPRLR 9.9 (SLICK2) | 0 |
| btPRLR 9.10 (SLICK2) | 0 |
| btPRLR 9.11 (SLICK3) | 0 |
| btPRLR 9.12 (SLICK3) | 15.9 |
| btPRLR 9.13 (SLICK3) | 6.8 |
| btPRLR 9.14 (SLICK3) | 0 |

### Example 3

### Surveyor Mutation Detection and RFLP Analysis

Sample preparation: Transfected cells populations at day 3 and 10 were collected from a well of a 6-well dish and 10-30% were resuspended in 50 μl of 1×PCR compatible lysis buffer: 10 mM Tris-Cl pH 8.0, 2 mM EDTA, 0.45% Tryton X-100(vol/vol), 0.45% Tween-20(vol/vol) freshly supplemented with 200 μg/ml Proteinase K. The lysates were processed in a thermal cycler using the following program: 55° C. for 60 minutes, 95° C. for 15 minutes. Colony samples from dilution cloning were treated as above using 20-30 μl of lysis buffer.

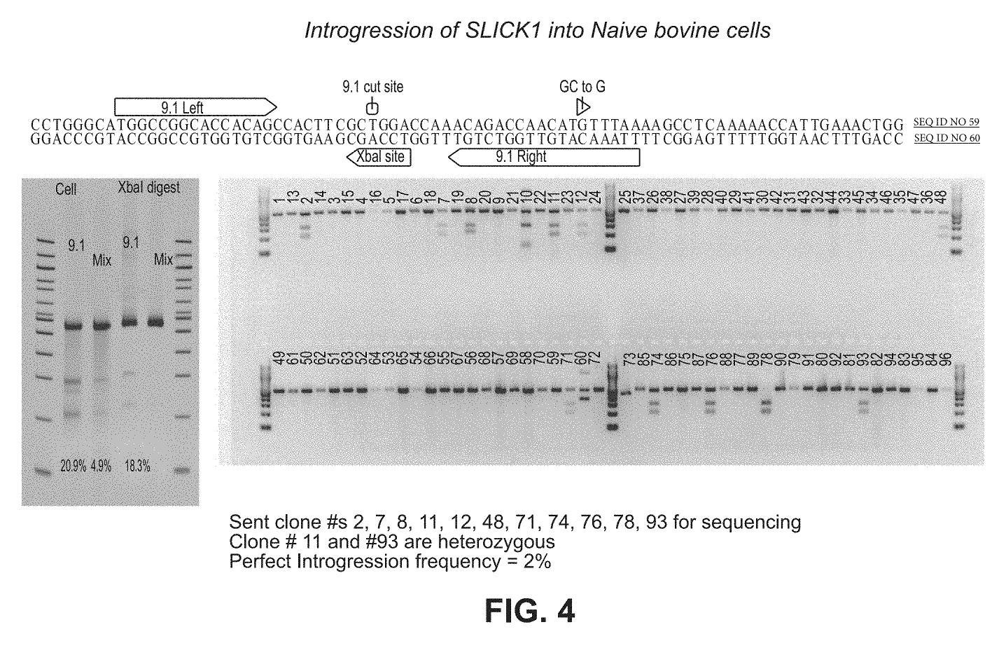

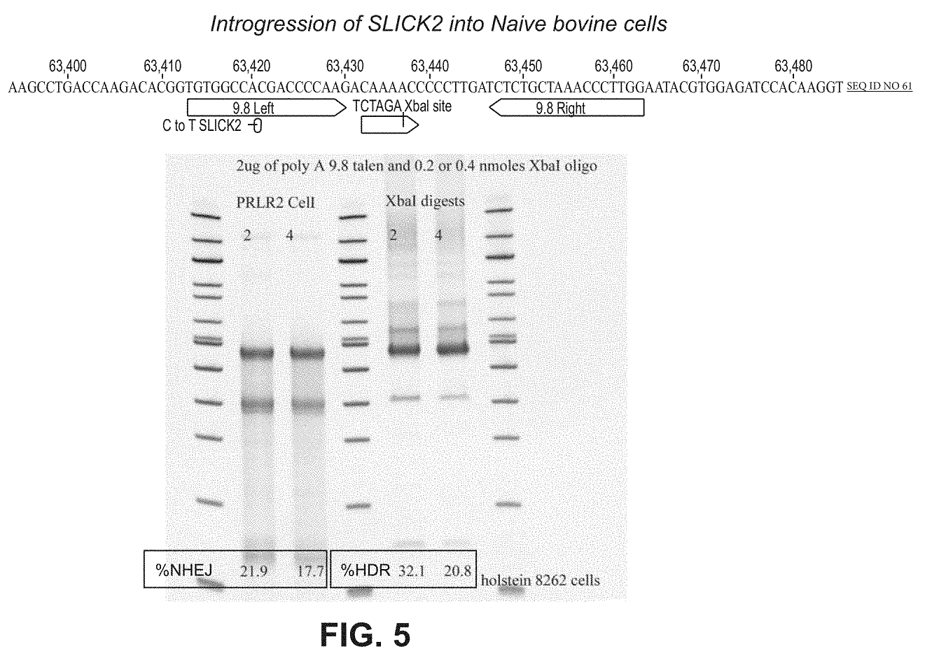

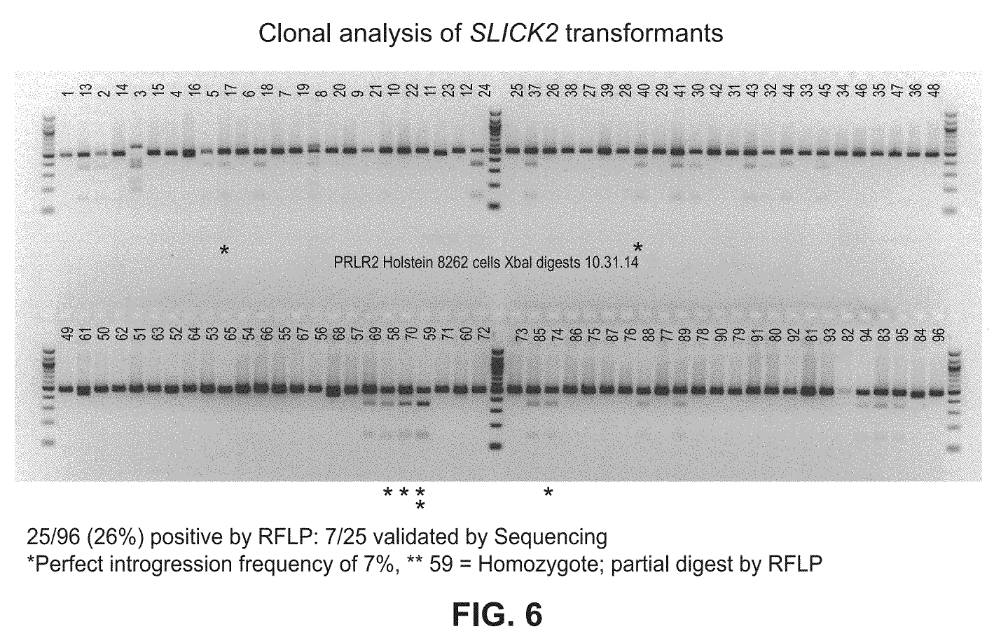

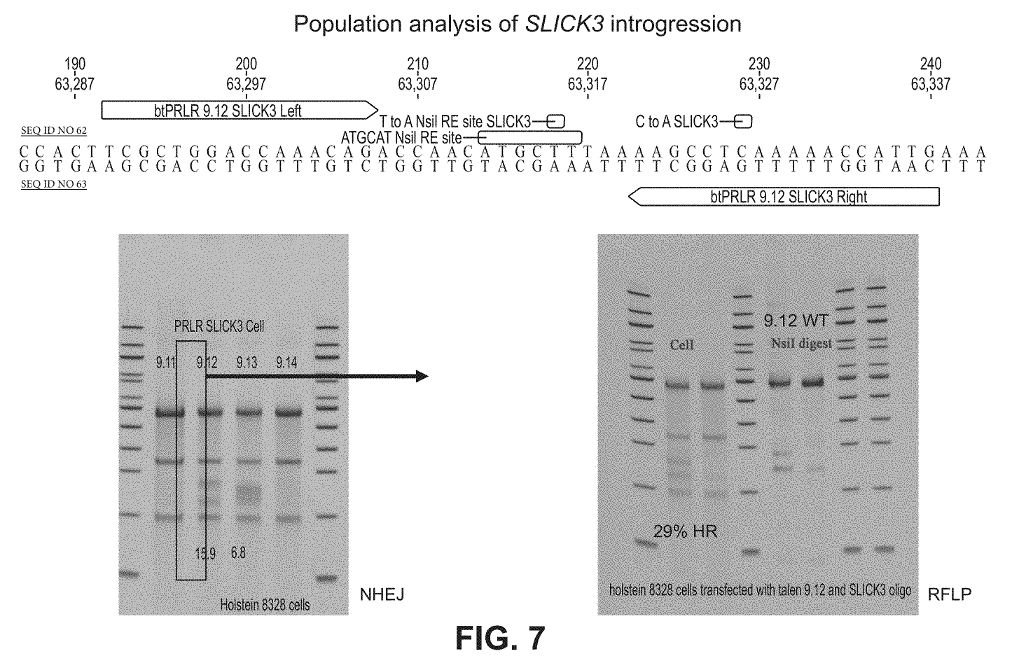

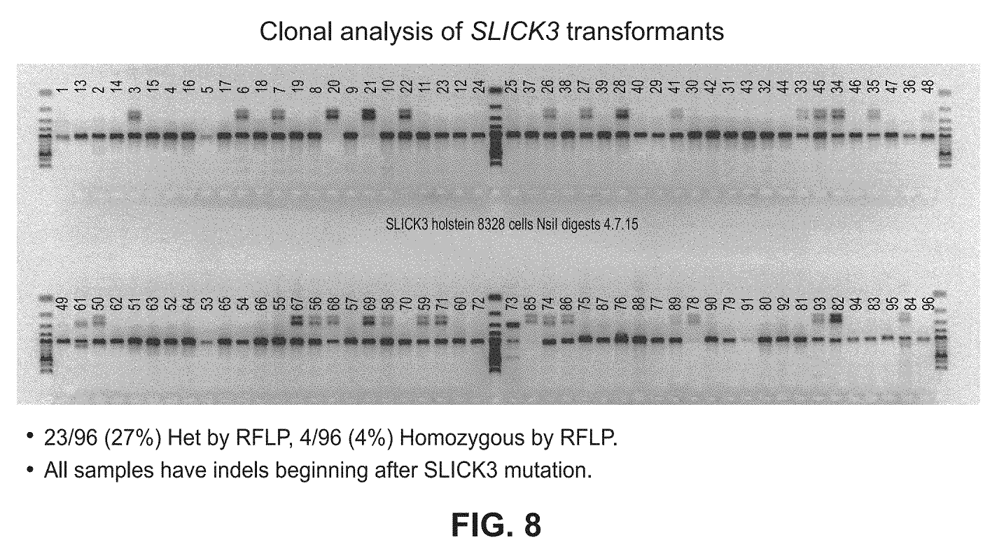

PCR flanking the intended sites was conducted using Platinum Taq DNA polymerase HiFi (Life Technologies) with 1 μl of the cell lysate according to the manufacturer's recommendations. Primers for each site are listed in Table IV. The frequency of mutation in a population was analyzed with the Surveyor Mutation Detection Kit (Transgenomic) according to the manufacturer's recommendations using 10 ul of the PCR product as described above. RFLP analysis was performed on 10 μl of the above PCR reaction using the indicated restriction enzyme. Surveyor and RFLP reactions were resolved on a 10% TBE polyacrylamide gels and visualized by ethidium bromide staining. Densitometry measurements of the bands were performed using ImageJ; and mutation rate of Surveyor reactions was calculated as described in Guschin et al. 2010(4). Percent HDR was calculated via dividing the sum intensity of RFLP fragments by the sum intensity of the parental band+RFLP fragments. For analysis of restriction site incorporation, small PCR products spanning the target site were resolved on 10% polyacrylamide gels and the edited versus wild type alleles could be distinguished by size and quantified. RFLP analysis of colonies was treated similarly except that the PCR products were amplified by 1×MyTaq Red Mix (Bioline) and resolved on 2.5% agarose gels. FIG. 4 illustrates, at top, the strategy for TALENs introduction of the SLICK1 mutation and introduction of the unique Xbal restriction site; bottom portion are gels showing RFLP analysis of SLICK1 transfected cells. FIG. 5, top, introgression strategy for introducing SLICK2 mutation into bovine cells and introduction of the unique Xbal site, bottom, agarose gel of colony mixture showing presence of Xbal restriction site. FIG. 6 is an agarose gel showing RFLP analysis of individual clones of the SLICK2 transformants. FIG. 7, top shows introgression strategy for introducing the SLICK3 mutation into bovine cells. Left gel is a mixture of colonies from treatment 9.11, 9.12, 9.13 and 9.14 (left to right), right gel confirmation of introgression showing endonuclease activity by Nsil activity. FIG. 8 is an agarose gel showing results of RFLP analysis of individual clones. The sequence of the TALENs RVDs are provided in the sequence listing accompanying this disclosure.

For the purposes of introgression of the SLICK phenotype into Red Angus genetics, 8 adult fibroblast lines were derived from elite female germplasm (TABLE V). Using the methods of SLICK1 introgression, (btPRLR9.1+ssODN, SEQ ID 44 or 45) were co-transfected into the cells which were analyzed for NHEJ and HDR at day 3 preceding colony production (TABLE V). The process has begun for 4 of the 8 lines and will continue to completion prior to cloning the modified cells to produce Red Angus animals with the SLICK phenotype.

TABLE IV
Primer pairs for RFLP analysis of introgression.

| | Primer Forward | Primer Reverse |
|---|---|---|
| Site | 5′ to 3′ | 5′ to 3′ |
| SLICK1 | ACCTTACATGTCTCCAGGCC | GGGACACCTTTGAGTACTCCT |
| | Seq ID 50 | Seq ID 51 |
| SLICK2 | ACCTTACATGTCTCCAGGCC | GGGACACCTTTGAGTACTCCT |
| | Seq ID 52 | Seq ID 53 |
| SLICK3 | ACCTTACATGTCTCCAGGCC | GGGACACCTTTGAGTACTCCT |
| | Seq ID 54 | Seq ID 55 |

TABLE V
Introgression of SLICK1 into elite Red Angus Germplasm

| Line ID | Day 3 RFLP | Day 3 CelI | Colony RFLP |
|---|---|---|---|
| 0545-X723 | 4.62% | 7.62% | 9/300 hets |
| D607 | 8.70% | 20.70% | 22/400 hets |
| C61 | not determined | not determined | Pending |
| C107 | not determined | not determined | Pending |
| C122 | Pending | Pending | Pending |
| C312 | Pending | Pending | Pending |
| C97 | Pending | Pending | Pending |
| B427 | Pending | Pending | Pending |

### Example 4

### Production of Animal Clones Expressing Slick Mutations

Upon confirmation of stable SLICK mutations described above in a bovine genome, somatic cell nuclear transfer, is used to produce a cloned animal expressing the mutation. Briefly, a transgenic bovine cell (or other artiodactyl if desired) such as an embryonic blastomere, fetal fibroblast, adult fibroblast, or granulosa cell that includes a nucleic acid mutation described above, is introduced into an enucleated oocyte to establish a combined cell. Oocytes can be enucleated by partial zona dissection near the polar body and then pressing out cytoplasm at the dissection area. Typically, an injection pipette with a sharp beveled tip is used to inject the transgenic cell into an enucleated oocyte arrested at meiosis 2. In some conventions, oocytes arrested at meiosis-2 are termed “eggs.” After producing a bovine (or other artiodactyl) embryo (e.g., by fusing and activating the oocyte), the embryo is transferred to the oviducts of a recipient female, about 20 to 24 hours after activation or up to 8 days after activation in cattle. See, for example, Cibelli et al. (1998) Science 280, 1256-1258 and U.S. Pat. No. 6,548,741. Recipient females can be checked for pregnancy starting at 17 days after transfer of the embryos.

### Example 5

### Production Cattle Expressing Slick Mutations by Embryo Microinjection

SLICK mutations have been engineered into bovine embryos directly, specifically for SLICK2 and SLICK3 sites (Table VI). Briefly, in vitro matured, in vitro fertilized bovine zygotes were injected with a combination of TALENs and repair template 14-24 hours post fertilization. Injection was directly into the cytoplasm of the zygote; TALEN mRNA and ssODN (HDR template) concentrations are listed in Table VI. Blastocyst formation rate (7 days post fertilization) did not differ significantly between buffer injected and TALENs-injected zygotes. Each condition was successful at producing embryos with INDEL mutations mediated by NHEJ, and precise HDR was observed in 5-19% of embryos. Total mutation rate was highest in SLICK2 injected embryos (>50% NHEJ+HDR), however, the frequency of precise introgression by HDR was higher for SLICK3. Considering the high mutation rates and unaffected embryo development, transfer of like produced embryos into surrogate dams, as in Example 4, is likely to produce cattle with the SLICK phenotype at high efficiency.

TABLE VI
SLICK2 and SLICK3 mutations in microinjected bovine zygotes.

| | Non- | Buffer- | | | |
|---|---|---|---|---|---|
| | injected | injected | TALENs-injected | | |
| | Blasto- | Blasto- | Blasto- | | |
| | cyst rate | cyst rate | cyst rate | NHEJ | HDR |
| | (%) | (%) | (%) | (%) | (%) |
| SLICK3 mutation | | | | | |
| mRNA btPRLR9.12 | 31.3 | 33.3 | 26.2 | 12.5 | 5 |
| 25 ng/μl | | | | | |
| ssODN (Seq ID 48) | | | | | |
| 100 ng/μl | | | | | |
| mRNA btPRLR9.12 | 37.8 | 27.1 | 22.1 | 7.31 | 19.51 |
| 40 ng/μl | | | | | |
| ssODN (Seq ID 48) | | | | | |
| 100 ng/μl | | | | | |
| SLICK2 mutation | | | | | |
| mRNA btPRLR9.8 | 33.1 | 17.2 | 24.9 | 42.1 | 10.5 |
| 40 ng/μl | | | | | |
| ssODN (Seq ID 46) | | | | | |
| 100 ng/μl | | | | | |

### Example 6

### Identification of Haplotype Markers Confirming Introgression of Slick Phenotype

The “SLICK” locus has been mapped to chromosome 20 of the cattle genome and the causative mutation underlying the phenotype for thermo-tolerance resides within the prolactin receptor (PRLR). The gene has nine exons that code for a polypeptide of 581 amino acids. Previous research in Senepol cattle has shown that the phenotype results from a single base deletion in exon 10 (there is no exon 1, recognized exons are 2-10) that introduces a premature stop codon (p.Leu462) and loss of the terminal 120 amino acids from the receptor. This phenotype is referred to herein as SLICK1. Senepol cattle are extremely heat tolerant and have been crossed with many other cattle breeds to provide the benefit of heat tolerance.

Table VII, below provides a marker analysis of SNPs around the SLICK locus. As shown, markers 1-5 are upstream of the SLICK locus on chromosome 20 and markers 6-10 are downstream of the SLICK locus. The row labeled “SNP Allele” is the locus on the chromosome where the markers (SNP) are found naturally in Senepol cattle. The row labeled “Other Allele” is the nucleotide residue of higher minor allele frequency among haired cattle and not found in the haplotype linked or containing SLICK. MAF is the frequency of each SNP compared to the WT within an experimental set of genotyped DNAs. The last column shows that the probability of having the SNP allele in the 10 flanking markers and not having the slick mutation is about 8×10⁻⁵. However, it should be noted that the sampling of animals for this study was heavily biased toward cattle DNA samples derived from animals influenced by a Criollo genetic base, the native sources of SLICK mutations. Therefore, the frequency of each of the markers is much more prevalent than it would be in any global/random distribution of these markers. The chance that a non-Senepol animal exhibited the deletion at Chr20-39136558 without having any of the linked markers would be 8×10−5 and this value is skewed to be more probable due to the sampling of a heavily influenced Criollo population. As noted in Table VII, the total length of the validation region is 296,033 bp, from 39,047,501 to 39,343,534.

TABLE VII

| Serial | | | | | | |
|---|---|---|---|---|---|---|
| Marker | 1 | 2 | 3 | 4 | 5 | Slick |
| SNP | Chr20- | Chr20- | Chr20- | Chr20- | Chr20- | Chr20- |
| | 39047501 | 39067164 | 39107872 | 39118063 | 39126055 | 39136558 |
| MAF | 0.425 | 0.419 | 0.424 | 0.422 | 0.322 | |
| SNP Allele | G | A | C | G | G | DEL (Slick) |
| Other Allele | T | G | T | A | T | C |

| Serial | | | | | | | |
|---|---|---|---|---|---|---|---|
| Marker | Slick | 6 | 7 | 8 | 9 | 10 | total = 10 |
| SNP | Chr20- | Chr20- | Chr20- | Chr20- | Chr20- | Chr20- | Prob by |
| | 39136558 | 39179498 | 39179527 | 39235859 | 39343400 | 39343534 | chance |
| MAF | | 0.397 | 0.412 | 0.276 | 0.423 | 0.423 | 8.28733E−05 |
| SNP Allele | DEL (Slick) | T | G | G | T | T | SLICK |
| | | | | | | | Haplotype |
| Other Allele | C | C | C | A | C | C | |

MAF = minor allele frequency;
SNP = single nucleotide polymorphism and is denoted by the coordinate position of the SNP on Chr 20 assembly of UMD 3.1 version of the bovine genome. Row designated SNP allele refers to the SNP allele represented in the SLICK Haplotype for the variant derived from Carribbean criollo cattle (i.e. the SLICK causative mutation found in Senepol cattle). Other allele represents the alternative SNP at this position as detected by the marker kit. All SNP listed in this table are bi-allelic. The probability of having the SNP allele in the 10 flanking markers and not having the SLICK mutation is about 8 × 10⁻⁵.

TABLE VIII
identifies the major haplotypes identified by the
markers of Table VII.
Table VIII

| SNP/Marker | Haplotype¹ | Haplotype Count |
|---|---|---|
| SLICK | GACGG-(Del)-TGGTT | 0.541 (n = 915) |
| WT | TGTAT-C-CCACC | 0.213 (n = 360) |
| 8 | TGTAT-C-CCGCC | 0.089 (n = 151) |
| 5 | TGTAG-C-CCACC | 0.029 (n = 49) |
| 5/8 | TGTAG-C-CCGCC | 0.027 (n = 46) |
| 5/6/7 | TGTAG-C-TGACC | 0.018 (n = 30) |
| 8/9/10 | TGTAT-C-CCGTT | 0.018 (n = 22) |
| | Other Haplotypes | 0.070 (n = 119) |
| | (<0.01) | |

Seven main haplotypes were identified in the SLICK validation region. As shown in Table 2, the first two haplotypes are SLICK and the WT.

Thus, once reliable markers are identified, the ability to further identify the source of a target sequence (SLICK as in Table VII) follows. In the case of SLICK, there have not been identified any haplotypes having the deletion of the cytosine base that do not also share all the alleles of the SLICK haplotype. Therefore, the chance that an animal from any population would have the cytosine deletion and not have the 10 other markers identified is so exceedingly low as to be impossible.

While this invention has been described in conjunction with the various exemplary embodiments outlined above, various alternatives, modifications, variations, improvements and/or substantial equivalents, whether known or that are or may be presently unforeseen, may become apparent to those having at least ordinary skill in the art. Accordingly, the exemplary embodiments according to this disclosure, as set forth above, are intended to be illustrative not limiting. Various changes may be made without departing from the spirit and scope of the invention. Therefore, the invention is intended to embrace all known or later-developed alternatives, modifications, variations, improvements and/or substantial equivalents of these exemplary embodiments.

The following paragraphs enumerated consecutively from 1 through 73 provide for various additional aspects of the present invention. In one embodiment, in a first paragraph, 1:

1 The present disclosure provides a livestock animal genetically modified to express a prolactin receptor (PRLR) gene resulting in a truncated PRLR.

2. The livestock animal of paragraph 1, wherein the PRLR is truncated after the tyrosine at residue 433 of the residue identified by GenBank Accession No. AAA51417.

3. The livestock animal of any of paragraphs 1 and 2, wherein the PRLR is truncated after the residue at AA 461.

4. The livestock animal of any of paragraphs 1 through 3, wherein the PRLR is truncated after the residue at AA 496.

5. The livestock animal of any of paragraphs 1 through 4, wherein the PRLR is truncated after the residue at AA 464.

6. The livestock animal of any of paragraphs 1 through 5, wherein the animal is less susceptible to heat stress.

7. The livestock animal of any of paragraphs 1 through 6, wherein the animal is an artiodactyl.

8. The livestock animal of any of paragraphs 1 through 7, wherein the artiodactyl is a bovine.

9. The livestock animal of any of paragraphs 1 through 8, wherein the genetic modification is made by nonmeiotic introgression.

10. The livestock animal of any of paragraphs 1 through 9, wherein the genetic modification is made by CRISPR/CAS, zinc finger nuclease, meganuclease, or TALENs technology.

11. The livestock animal of any of paragraphs 1 through 10, wherein the genetic modification is heterozygous.

12. The livestock animal of any of paragraphs 1 through 11, wherein the genetic modification is homozygous.

13. The livestock animal of any of paragraphs 1 through 12, wherein the PRLR gene is modified following residue 1383 of the mRNA as identified by GenBank Accession No. NM_001039726.

14. The livestock animal of any of paragraphs 1 through 13, wherein the modification results in a break in protein synthesis of the gene.

15. The livestock animal of any of paragraphs 1 through 14, wherein the animal expresses the SLICK phenotype.

16. A livestock animal genetically modified to express a SLICK phenotype comprising modification of the PRLR gene after residue 1383 as identified by the mRNA having GenBank accession No. NM_001039726.

17. The livestock animal of paragraph 16, wherein the modification is nonmeiotic introgression made by CRISPR/CAS, zinc finger nuclease, meganuclease, or TALENs technology.

18. The livestock animal of any of paragraphs 16 and 17, wherein the genetic modification results in a PRLR having between 433 amino acids and 511 amino acids as identified by GenBank Accession No. AAA51417.

19. The livestock animal of any of paragraphs 16 through 18, wherein the genetic modification results in a PRLR protein having 433 amino acids.

20. The livestock animal of any of paragraphs 16 through 19, wherein the genetic modification results in a PRLR protein having 461 amino acids.

21. The livestock animal of any of paragraphs 16 through 20, wherein the genetic modification results in a PRLR having 464 amino acids.

22. The livestock animal of any of paragraphs 16 through 21, wherein the genetic modification results in a PRLR having 496 amino acids.

23. The livestock animal of any of paragraphs 16 through 22, wherein the genetic modification results in a PRLR having 511 amino acids.

24. The livestock animal of any of paragraphs 16 through 23, wherein the modification is made to a somatic cell and the animal is cloned by nuclear transfer from the somatic cell to an enucleated egg.

25. The livestock animal of any of paragraphs 16 through 24, wherein the modification comprises a mutation that breaks protein synthesis by providing in a deletion, insertion or mutation of the genetic reading frame.

26. A method of genetically modifying livestock animals to express a SLICK phenotype comprising, expressing a prolactin receptor (PRLR) gene modified to break synthesis of the prolactin receptor (PRLR) protein after amino acid residue 433 as identified by GenBank Accession No. AAA51417.

27. The method of paragraphs 26, wherein the modification is made by providing a TALENs pair and a homology directed repair (HDR) template homologous to a portion of the PRLR designed to introduce a frame shift mutation or stop codon.

28. The method of any of paragraphs 26 and 27, wherein the break of synthesis is introduced after nucleotide 1383 of mRNA identified by GenBank accession No. NM_001039726.

29. The method of any of paragraphs 26 through 28, wherein the modification is made by CRISPR/CAS technology using guide RNA.

30. The method of any of paragraphs 26 through 29, further including introducing a nuclease restriction site proximate to the genetic modification.

31. The method of any of paragraphs 26 through 30, wherein the nuclease restriction site is downstream from the genetic modification.

32. The method of any of paragraphs 26 through 31, wherein the genetic modification and the introduction of the nuclease restriction site are directed by the same HDR template.

33. The method of any of paragraphs 26 through 32, wherein the genetic modification and the introduction of the nuclease restriction site are directed by different HDR templates.

34. The method of any of paragraphs 26 through 33, wherein the genetic modification is made to a somatic cell and the nucleus of the somatic cell is transferred to an enucleated egg of the same species.

35. The method of any of paragraphs 26 through 34, wherein the enucleated egg is renucleated and is transferred to a surrogate mother.

36. A genetically modified livestock animal according to any of the preceding paragraphs comprising a PRLR allele converted to express a SLICK phenotype.

37. A livestock animal cell comprising a genetically modified prolactin receptor (PRLR) allele resulting in a truncated PRLR.

38. The livestock animal cell of paragraph 37, wherein the PRLR is truncated after the tyrosine at residue 433 of the protein identified by GenBank Accession No. AAA51417.

39. The livestock animal cell of any of paragraphs 37 or 38, wherein the PRLR is truncated after the alanine residue at AA 461.

40. The livestock animal cell of any of paragraphs 37 through 39, wherein the PRLR is truncated after the proline residue at 496.

41. The livestock animal cell of any of paragraph 37 through 40, wherein the PRLR is truncated after the alanine residue at 464.

42. The livestock animal cell of any of paragraph 37 through 41, wherein the animal is less susceptible to heat stress.

43. The livestock animal cell of any of paragraphs 37 through 42, wherein the animal is an artiodactyl.

44. The livestock animal cell of any of paragraph 37 through 43, wherein the artiodactyl is a bovine.

45. The livestock animal cell of any of paragraphs 37 through 44, wherein the genetic modification is made by nonmeiotic introgression.

46. The livestock animal cell of any of paragraphs 37 through 45, wherein the genetic modification is made by CRISPR/CAS, zinc finger nuclease, meganuclease, or TALENs technology.

47. The livestock animal cell of any of paragraphs 37 through 46, wherein the genetic modification is heterozygous.

48. The livestock animal cell of any of paragraphs 37 through 47, wherein the genetic modification is homozygous.

49. The livestock animal cell of any of paragraphs 37 through 48, wherein the PRLR gene is modified following residue 1383 of the mRNA as identified by GenBank Accession No. NM_001039726.

50. The livestock animal cell of any of paragraphs 37 through 49, wherein the PRLR is modified to be truncated between residue Y433 and Y512 of the peptide as identified by GenBank Accession No. AAA51417.

51. The livestock animal cell of any of paragraphs 37 through 50, wherein the modification results in a break in protein synthesis of the gene.

52. The livestock animal cell of any of paragraphs 37 through 51, wherein the animal expresses the SLICK phenotype.

53. A livestock animal cell genetically modified to express a SLICK phenotype comprising modification of the PRLR gene after residue 1383 as identified by the mRNA having GenBank accession No. NM_001039726.

54. The livestock animal cell of paragraph 53, wherein the modification is made by nonmeiotic introgression using CRISPR/CAS, zinc finger nuclease, meganuclease, or TALENs technology.

55. The livestock animal cell of any of paragraphs 53 or 54, wherein the genetic modification results in a PRLR having between 433 amino acids and 511 amino acids as identified by GenBank Accession No. AAA51417.

56. The livestock animal cell of any of paragraphs 53 through 55, wherein the genetic modification results in a PRLR protein having from 433 amino acids.

57. The livestock animal cell of any of paragraphs 53 through 56, wherein the genetic modification results in a PRLR protein having 461 amino acids.

58. The livestock animal cell of any of paragraphs 53 through 57, wherein the genetic modification results in a PRLR having 464 amino acids.

59. The livestock animal cell of any of paragraphs 53 through 58, wherein the genetic modification results in a PRLR having 496 amino acids.

60. The livestock animal cell of any of paragraphs 53 through 59, wherein the genetic modification results in a PRLR having 511 amino acids.

61. The livestock animal cell of any of paragraphs 53 through 60, wherein the modification is made to a somatic cell and the animal is cloned by nuclear transfer from the somatic cell to an enucleated egg.

62. The livestock animal cell of any of paragraphs 53 through 61, wherein the modification comprises a mutation that breaks protein synthesis by providing in a deletion, insertion or mutation of the genetic reading frame.

63. A method of genetically modifying livestock animal cells to have a SLICK genotype comprising, expressing a prolactin receptor (PRLR) gene modified to break synthesis of the prolactin receptor (PRLR) protein after amino acid residue 433 as identified by GenBank Accession No. AAA51417.

64. The method of paragraph 63, wherein the modification is made by providing a TALENs pair and a homology directed repair (HDR) template homologous to a portion of the PRLR designed to introduce a frame shift mutation or stop codon.

65. The method of any of paragraphs 63 or 64, wherein the modification is made by CRISPR/CAS technology using guide RNA.

66. The method of any of paragraphs 63 through 65, wherein the break of synthesis is introduced after nucleotide 1383 of mRNA identified by GenBank accession No. NM_001039726.

67. The method of any of paragraphs 63 through 66, further including introducing a nuclease restriction site proximate to the genetic modification.

68. The method of any of paragraphs 63 through 67, wherein the nuclease restriction site is downstream from the genetic modification.

69. The method of any of paragraphs 63 through 68, wherein the genetic modification and the introduction of the nuclease restriction site are directed by the same HDR template.

70. The method of any of paragraphs 63 through 69, wherein the genetic modification and the introduction of the nuclease restriction site are directed by different HDR templates.

71. The method of any of paragraphs 63 through 70, wherein the genetic modification is made to a somatic cell and the nucleus of the somatic cell is transferred to an enucleated egg of the same species.

72. The method of any of paragraphs 63 through 71, wherein the enucleated egg is renucleated and is transferred to a surrogate mother.

73. A genetically modified livestock animal cell comprising a PRLR allele converted to express a SLICK genotype.

All patents, publications, and journal articles set forth herein are hereby incorporated by reference herein; in case of conflict, the instant specification is controlling.

While this invention has been described in conjunction with the various exemplary embodiments outlined above, various alternatives, modifications, variations, improvements, and/or substantial equivalents, whether known or that are or may be presently unforeseen, may become apparent to those having at least ordinary skill in the art. Accordingly, the exemplary embodiments according to this invention, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents of these exemplary embodiments.

1-37. (canceled)

38. A method of genetically modifying a bovine cell, the method comprising:
obtaining a bovine cell; and
editing a prolactin receptor (PRLR) gene of the bovine cell such that the cell expresses a truncated prolactin receptor (PRLR) protein, wherein the truncated prolactin receptor protein is 464 or 496 amino acids in length.

39. The method of claim 38, wherein editing the prolactin receptor gene does not involve meiotic introgression.

40. The method of claim 38, wherein editing the prolactin receptor gene comprises implementing CRISPR, zinc finger nuclease, meganuclease, or TALEN technology.

41. The method of claim 38, wherein editing the prolactin receptor gene comprises contacting the bovine cell with a TALEN pair that targets the PRLR gene.

42. The method of claim 38, wherein editing the prolactin receptor gene comprises introducing into the bovine cell a homology directed repair (HDR) template homologous to a portion of the PRLR gene, wherein the HDR template is designed to introduce a stop codon at amino acid residue 465 of the peptide as identified by SEQ ID NO. 64.

43. The method of claim 38, wherein editing the prolactin receptor gene comprises introducing a stop codon at amino acid residue 465 of the peptide as identified by SEQ ID NO. 64.

44. The method of claim 38, wherein editing the prolactin receptor gene comprises introducing into the bovine cell a homology directed repair (HDR) template homologous to a portion of the PRLR gene, wherein the HDR template is designed to introduce a stop codon at amino acid residue 497 of the peptide as identified by SEQ ID NO. 64.

45. The method of claim 38, wherein the editing the prolactin receptor gene comprises introducing a stop codon at amino acid residue 497 of the peptide as identified by SEQ ID NO. 64.

46. The method of claim 38, further comprising introducing a unique, non-native nuclease restriction site into a genome of the bovine cell.

47. The method of claim 46, wherein the unique nuclease restriction site is downstream from a stop codon inserted by modification of the prolactin receptor gene.

48. The method of claim 47, wherein the stop codon and the nuclease restriction site are introduced by a single homology directed repair (HDR) template.

49. The method of claim 47, wherein the stop codon and the nuclease restriction site are introduced by different homology directed repair (HDR) templates.

50. The method of claim 38, wherein modifying the prolactin receptor gene comprises implementing CRISPR technology using guide RNA.

51. The method of claim 38, wherein the bovine cell, after editing, is heterozygous for the truncated prolactin receptor.

52. The method of claim 38, wherein the bovine cell, after editing, is homozygous for the truncated prolactin receptor.

53. The method of claim 38, wherein the bovine cell is a somatic bovine cell.

54. The method of claim 53, further comprising transferring a nucleus of the somatic bovine cell to an enucleated egg of the same species.
